(19) United States
(12) Patent Application Publication
(10) Pub. No.: US 2002/0044946 A1
(43) Pub. Date: Apr. 18, 2002

(54) 12-25 kDa bacterial proteins and their 116-58 kDa polymers useful in anti-tumor vaccines

(76) Inventor: Nikolay Kislitchkine, Moscow (RU)

Correspondence Address: SUGHRUE MION ZINN MACPEAK & SEAS PLLC, 2100 Pennsylvania Avenue, N.W., Washington, DC, 20037-3213, US

(21) Appl. No.: 09/859,377

(22) Filed: May 18, 2001

Related U.S. Application Data
(63) Continuation of application No. PCT/GB99/03852, filed on Nov. 18, 1999.

(30) Foreign Application Priority Data
Apr. 15, 1999 (GB) 9908663.9
Nov. 18, 1998 (RU) 98120511

(51) Int. Cl. A61K 39/02; C12P 21/02; C12N 1/21

(52) U.S. Cl. 424/190.1; 530/395; 435/69.3; 435/252.3

(57) ABSTRACT

The present invention relates to bacterial proteins, particularly bacterial glycoprotein polymers with a molecular weight of 116 to 158 kDa which exhibit unique immunogenic and biological activities and may be used in vaccines and in pharmaceutical compositions, e.g., for treating tumors, and as biological effectors, such as anti-proliferation agents and nucleases.

The present invention relates to bacterial proteins which exhibit immunogenic and biological activities, their use in vaccines and pharmaceutical compositions and as biological effectors, such as anti-proliferation agents and enzymes.

The developments of vaccines has been a significant factor in the reduction of deaths resulting from infection by pathogenic and highly pathogenic microorganisms. Microorganisms which are highly pathogenic may attribute their virulence to their ability to penetrate specific eukaryotic cells and to remain viable and reproduce within them, yet remain unrecognized in the host animal (e.g. human) for long periods of time, resulting in “slow”, “unrecognized” or “undiagnosed” infections. The action of these microorganisms on vital cells of the body results in cell death and consequent damage or failure in the organs and systems of the macroorganism. However, in many cases there still exists a need for effective vaccines against pathogenic microorganisms.

At present, live vaccines, which were developed and approved 50 or more years ago, are available against the causative agents of plague, brucellosis, tularaemia, tuberculosis and several other infections. However, long-term clinical application of these vaccines has revealed several significant defects:

(1) live attenuated vaccines generally produce a high level of short-term immunity (LVS vaccine against tularaemia is an exception), leading to inadequate animal or human protection against a specific pathogen after a certain period of time, thus necessitating repeat vaccination;

(2) live attenuated vaccines retain residual virulence, a consequence of which is the reactogenicity of vaccinations, leading to a marked increase in the incidence of general temperature increase after a given vaccine; and

(3) many live vaccines are rapidly excreted by animals and humans and are therefore unable to produce full T-cell immunity, the primary factor in protecting the body from particular intracellular infections.

However, at present, there is no replacement for them and no known methods which might enable the development of new vaccines free of the above noted defects. The present invention therefore seeks to provide novel antigens of bacteria which may be used for the purposes of vaccination (particularly live vaccines) and in particular as protective immunogens in the control or prevention of diseases caused by bacteria, particularly highly virulent intracellular bacteria.

Surprisingly it has now been found that a previously unrecognized glycoprotein exists on the surface of bacteria, which is specific to the bacterium on which it is present, and which has unique immunomodulating and biological properties. This glycoprotein is believed to be at least partially responsible for the ability of the bacteria to bind to eukaryotic cells of a host macroorganism, penetrate their cytoplasm and survive and reproduce inside those. Thus these glycoproteins allow the development of vaccines (particularly live vaccines) and diagnostic products for the purposes of controlling, preventing and identifying diseases, in particular those resulting from infection with highly virulent bacteria.

The glycoproteins are a new class of biologically active compounds which are referred to herein as “tolins”. Tolins are previously unidentified glycoprotein polymers made up of several monomers. The monomers are produced in the cytoplasm and pass into the periplasmic space. Here, in association with polysaccharides through non-covalent interactions a glycoprotein polymer is formed which has the structural form of a “heat-shock” glycoprotein polymer. This then passes into the capsule. The glycoprotein is thus present in the outer membrane and capsule of bacterial cells.

When used herein, “tolin” refers to the glycosylated polymeric structure. The monomers which make up the biologically active tolin polymer are referred to herein as the tolin monomers and may be glycosylated or unglycosylated. Polysaccharides which together with the monomers make up the glycosylated polymeric tolin are referred to herein as “tolin polysaccharides”.

Tolins are specific to the bacterium in which they are present and possess unique enzymatic, immunomodulatory and antiproliferative properties. These glycoproteins, which in polymeric form are antigenic and which are constituents of the capsule, are also found in the periplasmic space, are novel, and may be used in for example the manufacture of vaccines against the source and related bacteria.

Thus viewed from one aspect the present invention provides a bacterial protein monomer which has a molecular weight of 12 to 25 kDa, for example about 17 kDa, as assessed by denaturing SDS-PAGE disk electrophoresis (in a BNB buffer 0.5 M Tris-HCl, pH 6.8, 7% SDS, 30% glycerin, 1% bromophenol blue, 15% 2-mercaptoethanol) and which in naturally occurring form forms part of a bacterial glycoprotein polymer present in the capsule of a bacterial cell, preferably said monomer is glycosylated with at least the monosaccharides glucose, xylose, rhamnose and ribose; or a functionally-equivalent variant, or fragment or precursor thereof.

Preferably, the monosaccharide derivatives glucosamine and galactosamine are absent. The precise polysaccharide portion of the glycoproteins of the invention is variable. By gel separation under non-denaturing conditions, it has been established that the polysaccharide portion of tolins is not covalently attached. Generally, a ratio of protein:polysaccharide of 1:2 is observed, but depending on the method of isolation, the polysaccharide moiety may comprise between 0.01×(isolated by HPLC) and 2×(isolated using Sepharose G200 with subsequent purification on DEAE cellulose) the amount of protein which is present. Low polysaccharide levels however appear to result in low stability and thus methods in which higher polysaccharide contents are retained are preferred. Partially deglycosylated tolins retain functional activity.

Whilst unglycosylated tolins have been found to lose some of the functions ascribed to glycosylated tolins (e.g. nuclease, cytotoxic and immunoprotective activities described hereinafter), surprisingly this form exhibits DNA-binding activity (see Example 2.10). This activity is not observed with glycosylated tolins although it may be masked by the nuclease activity of that form. This provides a convenient means of assessing the level of glycosylation of the tolin and may also allow modification, delay or release of latent activity by controlling the extent of glycosylation. Unglycosylated monomers and polymers as described herein with DNA-binding properties form preferred aspects of the invention.

It has furthermore been observed that the polymeric glycoproteins of the invention after isolation are not associated with any lipid components, as evidenced by gas chromatography studies (see Example 2.3) and the resistance of the glycoproteins to treatment with chloroform (see Example 2.3). The polymeric glycoproteins with a low polysaccharide content are however hydrophobic as exhibited by their behaviour during high pressure chromatography.

The tolin monomers (which have a molecular mass of between 12 and 25 kDa ) are hydrophobic, as indicated in HPLC and also by decoding the sequence of the first 45 amino acids. This has been confirmed by determining the entire 145 amino acid sequence. On loss of secondary and tertiary structure, which occurs when pH is adjusted, the polymeric tolin is also hydrophobic.

As used herein, “functionally-equivalent” defines proteins related to, or derived from, a naturally occurring bacterial protein monomer as defined herein, where the amino acid sequence has been modified by single or multiple amino acid substitution, addition and/or deletion and/or where the monomer is glycosylated, the extent or type of glycosylation has been altered, but which nonetheless retains functional activity.

Naturally occurring bacterial protein monomers, which may be glycosylated, are those which are found (either in monomeric, dimeric, trimeric or polymeric form) on unmodified bacteria and which may be isolated therefrom, or which may be produced synthetically, e.g. by expression of an appropriate expression vector encoding at least the amino acid sequence of the protein, in an appropriate host. Such monomers may be isolated in glycosylated form or may be separate from tolin polysaccharides. As a consequence of the cell-killing effects of polymeric glycoproteins of the invention, not all hosts can support the expression of the glycoproteins in the polymeric form. Avirulent or pathogenic microorganisms, e.g. gram-negative bacteria have the mechanisms to survive expression of the polymeric glycoproteins of the invention and thus form preferred hosts for the synthetic generation of the bacterial proteins in monomeric or polymeric form. When using pathogenic microorganisms as hosts, the pathogenicity is generally lost, to be replaced by the virulence conferred by the insert introduced into the host.

Functional equivalents as generally described above (ie. related to or derived from naturally occurring bacterial protein monomers) include variants, derivatives, precursors and fragments which retain one or more of the functions described herein (ie. retain functional activity). The bacterial proteins of the invention, at least when present in the polymeric glycosylated form, have a number of different functions as described herein, such as the ability to raise host protective antibodies and/or functional immunity against the bacteria. They also behave in a cytokine-like manner insofar as they are able to produce an anti-proliferative effect and kill cells. They also exhibit enzymatic activity, ie. nuclease activity. The polymeric and monomeric unglycosylated form exhibits DNA-binding properties. Thus, in subsequently discussed applications, when reference is made to bacterial proteins of the invention and their functionally equivalent variants etc., depending on the application under discussion, only variants etc. which retain the function appropriate for performing that application, e.g. which retain protective antigenic properties for vaccine applications, are included within the scope of such variants etc.

Furthermore, as will be clear from the discussions herein, polymeric structures, comprising at least three, e.g. four, of the monomer proteins defined herein, are generally required to achieve the stated functional effects. In this case, functional equivalents of the bacterial monomer protein include those equivalents which form the same function as the unmodified monomer, ie. when formed into the polymeric structure would exhibit the desired functionality. (An exception to this is DNA-binding activity which is exhibited both by monomeric and polymeric forms.)

Within the meaning of “addition” variants are included amino and/or carboxyl terminal fusion proteins or polypeptides, comprising an additional protein or polypeptide fused to the bacterial protein monomer sequence.

Such functionally-equivalent variants mentioned above include natural biological variations (eg. allelic variants or geographical variations or allotypic variations within a species or strain) and derivatives prepared using known techniques. For example, functionally-equivalent proteins may be prepared either by chemical peptide synthesis or in recombinant form using the known techniques of site-directed mutagenesis, random mutagenesis, or enzymatic cleavage and/or ligation of nucleic acids. Functionally-equivalent variants according to the invention particularly include analogues in different bacterial genera, species or strains.

Derivatives of the bacterial proteins may be prepared by post-synthesis/isolation modification of the glycoprotein, without affecting functionality, e.g. certain glycosylation, methylation etc. of particular residues. As mentioned above, the level of glycosylation may affect function and thus should be assessed in relation to the particular activity which is desired.

Functionally-equivalent fragments according to the invention may be made by truncation, e.g. by removal of a peptide from the N and/or the C-terminal ends or by selection of an appropriate active domain region which retains its functionality (e.g. antigenic properties) due to appropriate secondary and tertiary folding, or by deglycosylation.

A precursor as described herein may be a larger protein which is processed, e.g. by proteolysis, to yield the bacterial glycoprotein monomer per se. Such precursors may take the form of zymogens, ie. inactive precursors of enzymes, activated by proteolytic cleavage. This term is also intended to include polymeric structures (including tolins) made up of the monomeric proteins of the invention, e.g. dimers, trimers or polymers with more than 3, e.g. 4, monomers. Indeed, bacterial proteins of the invention are preferably in polymeric form and the polymeric structure comprises at least 3, e.g. 4 monomers, for example between 6 and 10 monomers and has a molecular weight of 116 to 158 kDa as assessed by non-denaturing SDS-PAGE by disk electrophoresis. The polymeric structure is preferably glycosylated as described previously (see Example 2.2), e.g. when prepared by gel filtration. Polymers glycosylated with low levels of polysaccharide may be obtained by HPLC separation. Essentially unglycosylated polymers may be prepared under certain conditions on HPLC or by non-denaturing gel electrophoresis (see hereinafter). In contrast to the monomers, the polymers exhibit the functional properties described above, depending on their state of glycosylation. Dimers and trimers composed of the monomers similarly have been found to lack the functional and enzymatic properties described above where these have been tested.

Bacterial glycoproteins of the invention in polymeric form have been found to be thermolabile and are disrupted to their monomers and free polysaccharide by heating in BNB buffer for 1 minute in a waterbath as detected by SDS-PAGE disk electrophoresis (see Example 2.1). Monomers separated under non-denaturing conditions on SDS-PAGE (without attached polysaccharide) may be reconstituted after extraction from the gel (see Examples 1.2.3 and 2.10) and concentrated to form unglycosylated polymers with DNA-binding properties described herein.

Tolins behave as good antigens and immunization of rabbits results in specific high-titre antibodies being produced. The interaction of tolins (also referred to herein as the antigen complex) and antibodies is readily detected by immunoelectrophoresis in 1% agarose. Tolins possess a weak negative charge and therefore form an immunoprecipitate in the anode area. The interaction of tolins and antibodies of animal blood serum may also be detected by radial immunodiffusion according to the method of Ochterlony using 1% agarose in Tris-barbiturate buffer, pH 8.6, with 0.1% Triton X-100 (see Example 2.4).

Tolins are hydrophilic compounds when the protein and polysaccharide portions are present in equivalent amounts. They may be maintained in stable form in aqueous solution, but readily form specific aggregations. They are very sensitive to variations in pH, the presence of chemical additives, temperature, concentration and composition of the solution, changes in which lead to the loss of hydrophilicity and formation of solid precipitates with an irreversible loss of solubility and of functional properties. The optimum pH at which the functional activities are exhibited, e.g. nuclease activity, is 7.0 to 8.0.

The antigen complex (tolin) possesses a weakly negative charge and interacts with antibodies of the immune sera of animal.

Thus in a preferred aspect the invention relates to a bacterial glycoprotein polymer which is comprised of at least three, e.g. four monomer proteins, which may be the same or different, wherein at least one monomer, preferably all monomers, are as described hereinbefore, to form a polymeric structure having a molecular weight of 116 to 158 kDa, for example a molecular weight in the range of 125 to 135 kDa, as assessed by non-denaturing SDS-PAGE, or a functionally-equivalent variant, derivative, fragment or precursor thereof. From the foregoing text it will be appreciated that separation by non-denaturing SDS-PAGE strips the polysaccharide portion from the tolin protein components. The glyco portion does not however affect the molecular weight significantly. Thus the molecular weight described above may alternatively be determined by gel filtration.

Preferably each polymeric glycoprotein has five or more monomeric units, e.g. 6 to 10 units, for example 6 units, which may be the same or different. In experiments which have been conducted certain preparations were found to have more than a single band in the region 116 to 158 kDa on SDS-PAGE. These preparations are also considered to fall within the definition of bacterial glycoprotein polymers described above.

The invention also extends to unglycosylated polymeric or monomeric forms, e.g. obtainable from a non-denaturing SDS-PAGE gel.

In the case of the bacterial protein (or glycoprotein) polymer, the functionally-equivalent variants etc. are as defined above for the monomer protein insofar as the functional activity is retained. Variants, derivatives, fragments or precursors may be produced by modification of one or more of the monomeric units, which may be the same or different, as defined above. The monomers of such a polymeric structure which may themselves not independently have the required activity are included within the scope of the invention as mentioned above.

The polymeric structures from the R-form of Francisella tularensis and from recombinant forms thereof into which DNA from a virulent bacteria had been inserted were purified, separated by denaturing SDS-PAGE, the 17 kDa bands isolated and partially sequenced. The protein moiety of the monomer of the bacterium (R-form Francisella tularensis) was found to have the amino acid sequence given below (see Example 2.7):

```
Met Glu Leu Lys Leu Glu Asn Lys Gln Glu Ile Ile Asp Gln Leu
                                    5 10 15
Asn Lys Ile Leu Glu Leu Glu Met Ser Gly Ile Val Arg Tyr Thr
                                  20 25 30
His Tyr Ser Leu Met ILe Ile Gly His Asn Arg Ile Pro Ile Val
                                  35 40 45
Trp Ser Met Gln Ser Gln Ala Ser Glu Ser Leu Thr His Ala Thr
                                  50 55 60
Ala Ala Gly Glu Met Ile Thr His Phe Gly Glu His Pro Ser Leu
                                  65 70 75
Lys Ile Ala Asp Leu Asn Glu Thr Tyr Gln His Asn Ile Asn Asp
                                  80 85 90
ILe Leu Ile Glu Ser Leu Glu His Glu Lys Lys Ala Val Ser Ala
                                  95 100 105
Tyr Tyr Glu Leu Leu Lys Leu Val Asn Gly Lys Ser Ile ILe Leu
                                110 115 120
Glu Glu Tyr Ala Arg Lys Leu Ile Val Glu Glu Glu Thr His Ile
                                125 130 135
Gly Glu Val Glu Lys Met Leu Arg Lys Tyr
                                140 145
```

The first 45 amino acids were also examined in recombinant bacteria in which DNA fragments derived from a virulent bacterium were inserted into the R-form Francisella tularensis (15NIIEG), RB7 and RM32 and found to be identical.

The above sequences have not been found to be closely related to any sequence in the MEL Protein database or other available databases, ie. they are less than 50% homologous to any know sequence, when assessed using the SWISS-PROT protein sequence databank using FASTA pep.cmp with a variable pamfactor, and gap creation penalty set at 12.0 and gap extension penalty set at 4.0.

Thus, alternatively viewed, the present invention provides bacterial protein monomers which contain the amino acid sequence:

```
Met Glu Leu Lys Leu Glu Asn Lys Gln Glu Ile Ile Asp Gln Leu
                                    5 10 15
Asn Lys Ile Leu Glu Leu Glu Met Ser Gly Ile Val Arg Tyr Thr
                                  20 25 30
His Tyr Ser Leu Met Ile Ile Gly His Asn Arg Ile Pro Ile Val
                                  35 40 45
Trp Ser Met Gln Ser Gln Ala Ser Glu Ser Leu Thr His Ala Thr
                                  50 55 60
Ala Ala Gly Glu Met Ile Thr His Phe Gly Glu His Pro Ser Leu
                                  65 70 75
Lys Ile Ala Asp Leu Asn Glu Thr Tyr Gln His Asn Ile Asn Asp
                                  80 85 90
Ile Leu Ile Glu Ser Leu Glu His Glu Lys Lys Ala Val Ser Ala
                                  95 100 105
Tyr Tyr Glu Leu Leu Lys Leu Val Asn Gly Lys Ser Ile Ile Leu
                                110 115 120
Glu Glu Tyr Ala Arg Lys Leu Ile Val Glu Glu Glu Thr His Ile
                                125 130 135
Gly Glu Val Glu Lys Met Leu Arg Lys Tyr
                                140 145
```

or a sequence which has more than 60%, preferably more than 80%, e.g. more than 90% sequence homology thereto (according to the test described above). Sequence identity at a particular residue is intended to include identical residues which have simply been derivatized.

Preferably bacterial proteins of the above sequence have the molecular weigth and/or glycosylation characteristics as described above.

In a preferred aspect, the invention extends to a bacterial protein polymer which is comprised of at least 3, e.g. 4 monomer proteins, which may be the same or different, wherein at least one monomer, preferably all monomers, are proteins containing the sequence defined above.

In addition, sequencing of the 17 kDa protein derived from SDS-PAGE yielded an additional sequence. In the preparation prepared from the R-form of Francisella tularensis, the following partial sequence was obtained:

```
Xxx Asn Arg Gly Ala Val Arg Lys Val Leu Thr Thr Gly Leu Xxx
                                          5 10
Ala Xxx Ile
  15
```

The residue at position 14 is believed to be a glutamic acid residue.

This sequence was present at a yield of approximately 17% relative to the main sequence. The 17 kDa preparation from a recombinant bacterium containing a DNA fragment from BCG (RB7) or melioidosis (RB32) contained a protein with the following partial sequence:

```
Xxx Asn Val Ser Glu Xxx Val Ser Ala Arg Ala Lys Glu Ala Asp
                                  5 10 15
Val Thr Xxx Glu Val Ala Ser Asn Thr Xxx Asp Ala Thr Ile Ala
                                  20 25 30
Ala Val Thr Xxx Ala Xxx Xxx Asn Xxx Xxx Ser Val Thr Leu Xxx
                                  35 40 45
Gly
```

The residue at positions 34, 36 and 39 are believed to be asparagine, leucine and arginine residues, respectively.

This sequence was present at a yield of 10-25% relative to the main sequence.

In the above sequences “Xxx” denotes unknown or variable residues which in the latter case may be any amino acid.

In addition to the above described proteins, when tolin-enriched extracts of the R-form of Francisella tularensis were separated on SDS-PAGE a band of approximately 12 kDa was identified. The N-terminal sequence of this protein was determined and is as follows:

```
Met Asn Lys Ser Glu Leu Val Ser Ala Ile Ala Lys Glu Ala Asp
                                            5 10 15
Val Thr Lys Glu Val Ala Ser Asn Thr Ile Asp Ala Thr Ile Ala
                                          20 25 30
Ala Val Thr Lys Ala Leu Lys Asn Gly Asp Ser
                                          35 40
```

This sequense is very similar to the sequence of a DNA-binding protein in the Swissprot database, HU:7/98 AC PO5384 and varies only at position 7 which has an isoleucine residue. This sequence was also found to be present in recombinant strains described herein. Although not wishing to be bound by theory this protein may be one or more of the monomers of polymers of the invention.

Proteins containing one or more of the above described sequences and sequences exhibiting more than 60%, preferably more than 80%, e.g. more than 90% sequence homology thereto (according to the test described above) form preferred aspects of the invention. Similarly the invention extends to a bacterial protein polymer which is comprised of at least 3, e.g. 4, monomer proteins, which may be the same or different, wherein at least one monomer contains one of the sequences described above.

The polymeric glycoproteins of the invention have also been found to have nuclease activity in vitro. This effect has been confirmed on both bacterial and eukaryotic DNA and tRNA in vitro. Thus the invention provides bacterial glycoprotein polymers as defined above which exhibit nuclease activity on DNA and RNA samples in vitro. As used herein, nuclease activity refers to the ability to cleave nucleic acid material e.g. as demonstrated by cleavage of 50% or more of said DNA over 60-90 minutes at 37° C. using DNA at a concentration of 0.8-1.2 μg/μl and bacterial glycoprotein polymer at a concentration of 0.8-1.6 μg/μl (see Example 2.5A and B) or cleavage of 50% or more of said RNA over 90-120 minutes at 37° C. using tRNA at a concentration of 1.5 to 2.0 μ/μl and bacterial glycoprotein polymer at a concentration of 0.8-1.6 μg/μl (see Example 2.5C). The polymeric glycoproteins have also been found to have cell killing or anti-proliferative properties as described hereinafter.

Furthermore, it has been found that the bacterial glycoprotein monomer described herein (and also the dimeric and trimeric form) is recognized by monoclonal antibodies to human tumour necrosis factor (TNFα). Three different series of monoclonal antibodies were used in the experiment, enabling interaction with the protein moieties of the tolin (see Example 2.4). However, these monoclonal antibodies were not characterized to determine whether they bound to the structural or functional part etc. of the antigens. Thus viewed in an alternative way, the invention provides bacterial glycoprotein monomers which bind to monoclonal antibodies directed to human TNFα. Furthermore, the bacterial glycoprotein in monomeric, dimeric and trimeric form is recognized by antibodies of normal blood sera of animals or man whereas the polymeric structure is not recognized by such sera.

The wide spectrum of binding of the monomer (and dimer and trimer forms) to monoclonal antibodies to TNF-α and normal blood sera antibodies suggests that this characteristic may be non-specific. This may be further corroborated by the fact that such antibodies do not bind to the polymeric structure.

It has also been found that bacteriophage, specific to the bacteria from which tolins of the invention are isolated, bind specifically to purified tolins. Thus tolins act as a receptor to bacteriophage. This may be explained by the DNA-binding activity of deglycosylated tolins described herein.

Purification of polymeric proteins of the invention has revealed that the polymeric complex elutes at 150 mM NaCl when chromatographed on a DEAE cellulose column in 10 mM Tris pH 7.5. Polymeric proteins of the invention were also found to elute at 51-52% acetonitrile when subject to gel filtration on a Nucleosil-C₁₈ column run in an acetonitrile/water mix with 0.1% TFA.

Bacterial protein polymers of the invention additionally exhibiting some or all of the structural or functional features described above form preferred aspects of the invention. For example, in a preferred feature, the invention provides a bacterial glycoprotein polymer which is comprised of at least 3, e.g. 4, monomer proteins, which may be the same or different, wherein at least one monomer, preferably all monomers, are as defined herein, to form a polymeric structure having a molecular weight of 116 to 158 kDa as assessed by non-denaturing SDS-PAGE, wherein said polymer elutes at 150 mM NaCl on DEAE cellulose and elutes at 51-52% acetonitrile on Nucleosil-C₁₈ and which exhibits nuclease activity in vitro. Furthermore, such polymers and/or their constitute monomers may have DNA-binding activity when unglycosylated.

The bacterial glycoprotein polymers of the invention are expressed on the surface of the bacteria (in naturally occurring and recombinant strains) as exhibited by bacteriophage binding studies. Bacterial protein (e.g. glycoprotein) monomers or polymers of the invention may thus be obtained by purification from lysates of the bacteria. Isolation of the pure bacterial proteins (e.g. glycoproteins) from the lysates may be performed, for example, by any of the following methods, HPLC, classic gel and ion-exchange chromatography or gradient ultracentrifugation. The crude extract of the bacteria may be prepared using conventional biochemical and surgical techniques, e.g. by homogenisation of the bacteria or other appropriate mechanism to disrupt its protein capsid/membrane envelope/cell wall in appropriate buffers, e.g. to prepare the lysate the bacteria may be homogenized using ultrasound without the application of detergents or other chemically biologically active components.

Thereafter the lysates may be clarified by centrifugation to remove intact cells and large fragments. The bacterial glycoprotein polymers of the invention may then be enriched in the preparation by adding (NH₄)₂SO₄ to 50% and subsequently adding (NH₄)₂SO₄ to 100% to salt out the protein. The precipitate (tolin-enriched lysate fraction) which is obtained may then be dialysed in 10 mM Tris-HCl buffer, pH 7.5, and may be used to recover tolins (which may be unglycosylated) in pure form according to the separation techniques indicated above, e.g. HPLC. At subsequent stages of further purification, conventional biochemical methods may be used providing all stages of recovery are conducted in the cold without the use of detergents and at a constant pH.

For example, the fraction of the tolin-enriched sample (obtained according to point 10 in Example 1, see gel chromatography) is acidified to pH 2.2 by adding trifluoroacetic acid (TFA). In the event of precipitation the sample is centrifuged at 12,000 rpm for 10 minutes. The supernatant is then applied to a column containing Nucleosil-C18 as packing (granule size 7 μm, pore diameter 10 Å) and the tolins (which may be unglycosylated) are separated by gradient elution using acetonitrile (from 0 to 70%). A “Gilson” HPLC apparatus is used.

Thus, a further aspect of the invention provides a method of preparing or isolating bacterial proteins (preferably glycoproteins) of the invention which comprises at least the step of subjecting a crude extract of bacteria to enrichment, e.g. by centrifugation (clarification) and ammonium sulphate precipitation, and recovering the bacterial protein polymer-containing fractions by an appropriate chromatographic technique or gradient ultracentrifugation. Preferably said method comprises at least the steps of (i) centrifugation (for clarification), (ii) ammonium sulphate precipitation of proteins of interest, (iii) size exclusion chromatography and (iv) ion-exchange chromatography (see Example 1).

The enriched tolin—(which may be unglycosylated) containing extracts may then be subject to further purification using conventional procedures e.g. centrifugation, selective precipitation, electrophoresis, chromatography and the like. Fractions containing the bacterial protein polymer of the invention may be identified by assays to identify, for example anti-proliferative/cell killing effects on fast-growing cells, nuclease activity, immuno electrophoresis with specific antibodies, e.g. Western blotting, binding to antibodies directed to human TNF, specific absorption of bacteriophages (see Example 2), and/or DNA-binding activity, depending on the state of glycosylation. The purity of the products may be determined by SDS-PAGE disk electrophoresis and the retention of secondary and tertiary structure by electron microscopy.

In order to obtain substantially unglycosylated polymers of the invention the polymer may be separated from polysaccharide after one or more of the steps described above, e.g. by the use of non-denaturing SDS-PAGE or HPLC under acid conditions. Polymer may be isolated directly or formulated from isolated unglycosylated monomers (see Examples 1.2.3 and 2.10). A method of preparing polymers of the invention from monomers as described herein forms a further aspect of the invention. Such a method comprises-at least the steps of purifying a glycoprotein polymer of the invention by appropriate techniques and separating the monomers from any remaining polysaccharide (e.g. by non-denaturing SDS-PAGE), isolating said monomers and concentrating said monomers, e.g. 10-fold or more, sufficient to allow formation of the polymeric structure.

As mentioned above, it has been found that the bacterial protein (e.g. glycoprotein) polymer acts as a receptor to bacteriophage directed to the bacteria. Such bacteriophage may therefore be used for identifying fractions containing the bacterial glycoprotein polymer of interest.

As an alternative to preparing crude lysate of the bacteria, the bacterial glycoprotein polymer may be released from the surface-of the bacteria in truncated form, e.g. by treatment with a proteolytic enzyme. Enzymes such as trypsin or endonuclease Glu-C are not useful in this respect if complete digestion is allowed since their products yield fragments without the characteristic activities of polymers of the invention.

An alternative preparation process may take advantage of the binding activities of the bacterial proteins by using an affinity chromatography system in which specific ligands are immobilised on a solid phase matrix. Suitable binding partners include bacteriophages and antibodies which bind to the bacterial proteins (preferably glycoproteins), e.g. antibodies raised by challenge with bacterial glycoproteins of the invention or antibodies to human TNF (depending on whether the monomeric or multimeric form is to be isolated).

Thus the invention also provides a method of preparing or isolating the bacterial proteins (preferably glycoproteins) of the invention, said method comprising at least the steps of preparing an extract of said bacteria, purifying said bacterial protein therefrom by binding said bacterial protein to an immobilized phase including a specific binding partner for the bacterial protein and subsequently eluting said bacterial protein from said immobilized phase.

Preferably, the bacterial proteins are isolated in their polymeric form, ie. their naturally occurring form. Example 1 provides an appropriate technique for performing this.

Bacterial proteins (preferably glycoproteins) obtainable by the methods described above form a further aspect of the invention.

In addition to the extraction and isolation techniques mentioned above, the bacterial proteins may be prepared by recombinant DNA technology using standard techniques, such as those described for example by Sambrook et al., 1989, (Molecular Cloning, a laboratory manual 2 nd Edition, Cold Spring Harbor Press).

Nucleic acid molecules comprising a nucleotide sequence encoding the bacterial proteins of the invention thus form further aspects of the invention. In one embodiment, the present invention thus provides a nucleic acid molecule encoding a bacterial protein of the invention, or a functionally-equivalent variant, derivative, fragment or precursor thereof as defined above.

Nucleic acid molecules according to the invention may be single or double stranded DNA, cDNA or RNA, preferably DNA, and include degenerate, substantially homologous and hybridising sequences which are capable of coding for the bacterial protein or bacterial protein fragment or precursor concerned. By “substantially homologous” is meant sequences displaying at least 60%, preferably at least 70% or 80% sequence homology. Sequence homology at a particular base is intended to include identical bases which have been derivatized. Hybridising sequences included within the scope of the invention are those binding under non-stringent conditions (6×SSC/50% formamide at room temperature) and washed under conditions of low stringency (2×SSC, room temperature, more preferably 2×SCC, 420° C.) or conditions of higher stringency eg. 2×SSC, 65° C. (where SSC=0.15 M NaCl, 0.015 M sodium citrate, pH 7.2), as well as those which, but for the degeneracy of the code, would hybridise under the above-mentioned conditions.

Derivatives of nucleotide sequences capable of encoding functionally-equivalent bacterial proteins, e.g. antigenically active bacterial proteins or bacterial protein variants according to the invention, may be obtained by using conventional methods well known in the art.

Bacterial proteins according to the invention may be prepared in recombinant form by expression in a host cell containing a recombinant DNA molecule which comprises a nucleotide sequence as broadly defined above, operatively linked to an expression control sequence, or a recombinant DNA cloning vehicle or vector containing such a recombinant DNA molecule. As mentioned above, not all hosts will tolerate expression of the polymeric bacterial proteins of the invention which on binding to a polysaccharide moiety acquire nuclease activity and thus preferably the host is an avirulent or pathogenic microorganisms, e.g. a gram-negative bacterium. Synthetic polypeptides expressed in this manner form a further aspect of this invention (the term “polypeptide” is used herein to include both full-length protein and shorter length peptide sequences).

The bacterial protein so expressed may be a fusion polypeptide comprising all or a portion of a bacterial protein according to the invention and an additional polypeptide coded for by the DNA of the recombinant molecule fused thereto. This may for example be β-galactosidase, glutathione-S-transferase, hepatitis core antigen or any of the other polypeptides commonly employed in fusion proteins in the art.

Other aspects of the invention thus include cloning and expression vectors containing the DNA coding for a bacterial protein of the invention and methods for preparing recombinant nucleic acid molecules according to the invention, comprising inserting nucleotide sequences (which when inserted into an appropriate eukaryotic or prokaryotic cell encodes the bacterial protein) into vector nucleic acid, eg. vector DNA. Such expression vectors include appropriate control sequences such as for example translational (eg. start and stop codons, ribosomal binding sites) and transcriptional control elements (eg. promoter-operator regions, termination stop sequences) linked in matching reading frame with the nucleic acid molecules of the invention.

Vectors according to the invention may include plasmids and viruses (including both bacteriophage and eukaryotic viruses) according to techniques well known and documented in the art, and may be expressed in a variety of different expression systems, also well known and documented in the art. Suitable viral vectors include baculovirus and also adenovirus and vaccinia viruses. Many other viral vectors are described in the art.

A variety of techniques are known and may be used to introduce such vectors into prokaryotic or eukaryotic cells for expression, or into germ line or somatic cells to form transgenic animals. Suitable transformation or transfection techniques are well described in the literature.

The invention also includes transformed or transfected prokaryotic or eukaryotic host cells, or transgenic organisms containing a nucleic acid molecule according to the invention as defined above. As mentioned previously, not all prokaryotic and eukaryotic cells will support expression of polymeric bacterial glycoproteins of the invention as a consequence of their cell-killing properties.

Only intracellular pathogenic bacteria (e.g. pathogens of tularaemia, snive (glanders), tuberculosis and other diseases) possess the appropriate cellular mechanisms for processing the unglycosylated polymer (or monomers) into a glycosylated polymer having nuclease activity with subsequent or simultaneous transfer into the periplasmic space and then capsule. This ability is absent in non-pathogenic bacteria (gut bacilli, bifid bacteria etc.) since they do not contain tolin-like monomers or polymers and are therefore unsuitable for expression of tolins. Similarly, eukaryotic cells are unsuitable. This is especially so as any tolins produced would kill surrounding non-tolin expressing cells.

For expression of the fully functional polymeric bacterial proteins therefore, expression is preferred in avirulent or pathogenic microorganisms, e.g. gram-negative bacteria. These hosts also provide the necessary mechanisms for assembly of the polymeric form of the bacterial protein. However, these problems do not exist with the expression of the bacterial protein monomers which do not exhibit nuclease activity or for the expression of polymers (or monomers) having DNA-binding activity. In such cases appropriate host cells may for example include prokaryotic cells such as E.coli, eukaryotic cells such as yeasts or the baculovirus-insect cell system, transformed mammalian cells and transgenic animals and plants.

It should be remembered when expressing proteins of the invention in bacterial cells that naturally occurring tolins specific to those cells pre-exist in the bacterial cells. However, by insertion of appropriate foreign DNA as described above, proteins of the invention distinct (e.g. in terms of reactivity to specific bacteriophages or antibodies) to the naturally occurring proteins are expressed.

A number of recombinant organisms have been made in which DNA fragments from pathogenic bacteria have been inserted into the commercially available R-form of Francisella tularensis (and deposited at the Russian National Collection of Industrial Microorganisms under deposit number VKPM B-6854). These recombinant microorganisms, which express bacterial proteins of the invention, have been deposited at the Russian National Collection of Industrial Microorganisms (VKPM) under the Budapest Treaty. RTC16 containing an insert from snive (glanders) bacteria, RRCC207 also containing an insert from the infectious agent responsible for snive, RM32 containing an insert from the bacteria responsible for melioidosis and RM28 also containing an insert from melioidosis bacteria were deposited on Nov. 16, 1998 in the name of Bioscan Ltd and given Accession numbers VKPM B-7673, VKPM B-7672, VKPM B-7671 and VKPM B-7670, respectively. In addition, Francisella tularensis subsp. Holarctica was used to form R5S containing an insert from Francisella tularensis nearctica Shu, RN4 containing an insert from Pseudomonas (Burkholderia) pseudomallei C-141, R1A containing an insert from Francisella tularensis nearctica B-399A Cole, were deposited at the same Depositary on Aug. 8, 1994 under Accession numbers VKPM B-6853, VKPM B-6855 and VKPM B-6852, respectively. Furthermore Francisella tularensis subsp. Holarctica was used to form RM2 containing an insert from melioidosis bacteria, RB7 containing an insert from tuberculosis bacteria, RB26 containing an insert from tuberculosis bacteria and RC117 containing an insert from snive (glanders) bacteria which were deposited at the same Depositary on Apr. 8, 1997 and RVT-1 and RVT-2 each containing inserts from tuberculosis bacteria, which were deposited at the same Depositary on May 7, 1999 under Accession numbers VKPM B-7381, VKPM B-7383, VKPM B-7382, VKPM B-7384, VKPM-7776 and VKPM-7775, respectively.

A further aspect of the invention provides a method for preparing or isolating a bacterial protein of the invention as hereinbefore defined, which comprises culturing a host cell containing a nucleic acid molecule encoding all or a portion of said bacterial protein, under conditions whereby said bacterial protein is expressed and recovering said bacterial protein thus produced. Preferably such cells are those which have been deposited and are described herein. Bacterial proteins of the invention may be isolated from such cells according to the method described in Example 1.

The bacterial proteins of the invention and functionally equivalent bacterial protein variants, derivatives, fragments or precursors thereof may also be prepared by chemical means, such as the well known Merrifield solid phase synthesis procedure.

Bacterial proteins according to the invention may be obtained from, or derived from the bacterial glycoprotein of, any pathogenic intracellular bacteria and their variants. Particularly preferred are gram negative and gram positive bacteria, especially bacteria of the genus Pseudomonas (Burkholderia) (e.g. P. mallei and P. pseudomallei) and the family Mycobacteriaceae (genus Mycobacterium; BCG, M. bovis, M. tuberculosis). Francisella (sp. F. tularensis, R-form of the vaccine strain 15 NIIEG) is particularly preferred.

It has been found that the bacterial glycoprotein polymers are effective immunomodulators which create both B-and T-cell responses, ie. which result in both humoral and cell immunity. Tolins are believed to be largely responsible for the virulence of pathogenic bacteria and for the first time this offers the possibility of preparing vaccines for bacteria for which this was not previously possible, or which relied on poor vaccine compositions such as attenuated bacteria. Antigens of the invention do not require attenuation and allow the use of live vaccines containing (and/or encoding) bacterial proteins of the invention which thereby allow the bacterial proteins of the vaccine to remain in the body for sufficient lengths of time to develop full immune responses.

Thus, in a further aspect, the invention provides a vaccine composition comprising one or more bacterial proteins (preferably glycoproteins) of the invention, preferably bacterial protein polymers, together with at least one pharmaceutically acceptable carrier, diluent or excipient. Furthermore, the invention provides the use of a bacterial protein of the invention, and functionally-equivalent variants, derivatives, precursors or fragments thereof, for the preparation of a vaccine composition for use in stimulating an immune response against said bacterium or a related bacterium (e.g. of the same genus) in a human or non-human animal. As mentioned previously, the glycoprotein polymer form has been found to exhibit various functions which are absent from the monomer and the unglycosylated polymer. Similarly, the glycoprotein polymer is advantageously used in the vaccine since this exhibits substantially greater protective effects than the monomer or unglycosylated polymer.

In a preferred aspect, said bacterial proteins are formed in a live vaccine, ie. they are produced in the body of the vaccinated animal e.g. human. This may be achieved by expression in a host cell which can self-replicate in the vaccinated body, e.g. use of a host microorganism such as a gram negative bacterium as described above.

It has been found that bacterial glycoprotein polymers of the invention derived from different bacteria exhibit specificity for that organism, as displayed by their reaction with immune sera prepared using live pathogenic bacteria as immunogens. However, the monomers described herein appear to be highly conserved in various bacteria and some cross-reactivity occurs. The bacterial glycoprotein polymers of the invention from different pathogenic bacteria have been found to offer protection against related bacteria. Thus bacterial proteins derived from different bacteria may be used as vaccines against infections resulting from that, or closely related, bacteria.

Further provided according to the invention is a vaccine composition for stimulating an immune response against a bacterium in a human or non-human animal comprising one or more bacterial proteins, or functionally-equivalent variants, derivatives, antigenic fragments or precursors thereof, as defined above, together with a pharmaceutically acceptable carrier or diluent, and a method of stimulating an immune response against a bacterium in a human or non-human animal, comprising administering to said animal a vaccine composition as defined above.

Preferably, the animal to be treated is a mammal, especially preferably a human.

As mentioned above, bacterial proteins according to the invention may be obtained from any bacterium. Preferably however, for use as vaccines, the bacterial proteins are obtained from, or derived from bacterial glycoproteins obtainable from gram negative intracellular bacteria, particularly of the genus Pseudomonas (Burkholderia), sp. F. tularensis and Mycobacterium, which are used to stimulate an immune response which is protective against these and related bacteria. Bacterial proteins which may be used to prepare vaccines against a range of bacteria, so called “broad spectrum” bacterial protein antigens (ie. which are capable of stimulating host protective immune responses against, in addition to the bacterium from which they were isolated, a broad range of other bacteria), are especially preferred. However, in developing a “broad spectrum” vaccine, it should be considered that the broader the spectrum of pathogenic bacteria against which a universal vaccine is developed, the lower the index of protection achieved against each specific virulent strain included in the spectrum.

Especially preferably, bacterial glycoproteins of the invention for use in vaccines are in the polymeric form, ie. having a molecular weight of between 116 and 158 kDa and comprising 4 or more monomers and contain a polysaccharide moiety present in a ratio of protein:polysaccharide of 1:1 or less (e.g. 1:2). As mentioned above, said bacterial proteins are preferably present in a live, self-replicating form, such as in a pathogenic microorganism. Single monomers have not been found to be effectively protective in the systems tested.

As referred to herein, bacterial proteins (e.g. present in a bacterium or other carrier) which are capable of stimulating an immune response, generate a host-protective, ie. immunogenic, immune response, that is a response by the host which leads to the generation of immune effector molecules, antibodies or cells which damage, inhibit or kill the bacterium, or related bacterium, and thereby protects the host from clinical or sub-clinical (ie. asymptomatic) disease. Such a protective immune response may commonly be manifested by the generation of antibodies and the development of delayed or immediate types of hypersensitivity able to suppress the metabolic functions of the bacterium.

As mentioned above, one of the ways in which the bacterial proteins of the invention may exert their host protective effects is by activation of the macroorganisms' immunity which inhibits the growth, maintenance and/or development of the bacterium, e.g. as exhibited by a maintenance or reduction in the numbers of pathogenic bacteria within the cells of the human or non-human animal.

Increasing the number of inhibitory serum antibodies does not always suppress the growth, vital activity and/or development of intracellular bacteria, but may be a highly specific diagnostic sign of the presence of pathogenic bacteria. Such antibodies and their antigen-binding fragments (eg. F(ab)_{2,} Fab and Fv fragments ie. fragments of the “variable” region of the antibody, which comprises the antigen binding site) which may be mono- or polyclonal, form a further aspect of the invention, as do vaccine compositions containing them and their use in the preparation of vaccine compositions for use in passively immunising hosts against bacteria. Such inhibitory antibodies may be raised by use of idiotypic antibodies. Anti-idiotypic antibodies may be used as immunogens in vaccines.

A vaccine composition may be prepared according to the invention by methods well known in the art of vaccine manufacture. Traditional vaccine formulations may comprise one or more antigens (bacterial proteins) or antibodies according to the invention together, where appropriate, with one or more suitable adjuvants eg. aluminium hydroxide, saponin, quil A, or more purified forms thereof, muramyl dipeptide, mineral or vegetable oils, Novasomes or non-ionic block co-polymers or DEAE dextran, in the presence of one or more pharmaceutically acceptable carriers or diluents. Suitable carriers include liquid media such as saline solution appropriate for use as vehicles to introduce the bacterial proteins of the invention into an animal or patient. Additional components such as preservatives may be included.

An alternative vaccine formulation may comprise a virus or host cell eg. a microorganism (eg. vaccinia virus, adenovirus, bacteria such as the Bacillus Calmette-Guérin strain of Mycobacterium bovis (BCG) or Salmonella spp) which may be live, killed or attenuated, having inserted therein a nucleic acid molecule (eg. a DNA molecule) according to this invention for stimulation of an immune response directed against polypeptides encoded by the inserted nucleic acid molecule. This method provides the advantage that the antigen (bacterial protein) may be continuously produced in the body thus allowing the development of a full immune response.

Especially preferably, vaccines comprise one of the deposited recombinant microorganisms mentioned herein or a tolin (preferably in glycosylated form) derived therefrom. In particular vaccines using the recombinant strains designated RE32 and RE28 or tolins purified therefrom (according to the method of Example 1) are preferred.

Administration of the vaccine composition may take place by any of the conventional routes, eg. orally, rectally or parenterally such as by intramuscular, subcutaneous, intraperitoneal or intravenous injection, optionally at intervals eg. two injections at a 7-35 day interval. Immunization by topical application of a composition, e.g. an ointment, to the skin is also possible.

The bacterial protein antigens may be used according to the invention in combination with other protective antigens obtained from the same or different bacteria. Such a combined vaccine composition may contain smaller amounts of the various antigens than an individual vaccine preparation, containing just the bacterial protein antigen in question.

Since the bacterial proteins of the invention exist on the surface of bacteria, their presence may be used to identify the presence of said bacteria. Clearly this has applications in the diagnosis of patients infected by such bacteria or may be used to identify the presence of bacteria in biological or non-biological samples, e.g. cell culture supernatants or in water samples to check for contamination.

Thus, the present invention further provides a method of identifying the presence, or determining the amount, of a bacterium or part thereof in a sample, comprising at least the step of assessing the presence or amount of a bacterial protein of the invention or fragment thereof or nucleic acid molecule encoding said protein or fragment thereof in said sample. As used herein, “part” refers to any portion of the bacterium which carries the bacterial protein of the invention or its encoding nucleic acid material or a fragment of the bacterial protein or its encoding nucleic acid material which would allow identification of said bacterial protein or its encoding nucleic acid material by one of the methods described herein. As used herein “fragment” refers to a portion of the protein which allows unique identification of the protein from which it is derived, e.g. a region of less than 100 residues, e.g. 5 to 20 residues. The term “assessing” as used herein includes both quantitation in the sense of obtaining an absolute value for the amount of bacteria in a sample, and also obtaining a semi-quantitative assessment or other indication, e.g. an index or ratio, of the amount of bacteria in the sample. Conveniently, to determine the amount of bacteria which are present, a standard curve relating the presence of bacterial protein (or encoding nucleic acid material) to the level of bacteria in a particular sample type may be prepared using control samples spiked with different amounts of said bacterium or part thereof. The test sample result may then be compared to the standard curve to determine the amount of bacteria which are present.

To perform the assessment step of the assay method, a technique which allows identification/visualization (signalling means) of the bacterial protein or fragment thereof or its encoding nucleic acid material must be performed. As discussed herein, the bacterial proteins of the invention have several unique properties, and these properties may be used as indicators of the presence of the bacterial proteins in the sample under study. The antigenic properties of the bacterial proteins may be utilized to prepare a marker for the presence of the bacterial proteins, e.g. antibodies, preferably monoclonal, directed to the particular bacterial protein (essentially unique to said bacterial protein to avoid high background levels) may be prepared and used.

To allow assessment of the amount of antibody bound to the bacterial protein, the antibodies may be provided with a label directly or indirectly. Such labels or means for labelling include for example, enzymes, fluorescent compounds, radio-labels and chemiluminescent compounds. A label which uses enzyme activity to generate a colour for spectrophotometric assessment may also be used, e.g alkaline phosphatase. To identify only those antibodies binding to the bacterial proteins, unbound antibodies should be removed and appropriate washing steps may be used for this purpose. For example, sandwich assays may be used in which the bacterium bearing the bacterial protein is immobilized on a solid support (e.g. via an antibody) and is then contacted with an antibody (bearing a label) directed to the bacterial protein. Unbound antibody (and hence label) may simply be washed away. Thus, the assay method of the invention may for example be performed as an ELISA.

Alternatively, different properties of the bacterial protein may be assessed. Thus, for example, the level of nuclease activity in a sample may be assessed as an indicator of the presence of the bacterial protein polymer. As mentioned previously, bacteriophage recognize and bind to bacterial protein polymers of the invention derived from the bacteria to which such bacteriophage are directed. Thus, bacteriophages may be used to identify the presence of bacterial protein polymers of the invention and hence bacteria in a sample (see Example 2.6).

Alternatively, DNA-binding activity may be assessed by stripping the polysaccharide moiety from the protein (e.g. by acid hydrolysis). The use of nucleic acid probes, e.g. DNA probes, complementary to the DNA sequence encoding one of the amino acid sequences described herein provides a further method of identifying bacterial DNA and hence the presence of that bacteria.

The invention furthermore extends to kits for performing the assay methods of the invention. Thus the present invention provides a kit for identifying the presence, or determining the amount, of a particular bacterium or part thereof in a sample, comprising at least the following:

i) a signalling means, e.g. a label-carrying antibody binding to a bacterial protein of the invention or fragment thereof, specific to said bacterium, or a substrate appropriate to the enzymatic activity of said bacterial protein, or a labelled nucleic acid probe which binds to a nucleic acid molecule encoding a bacterial protein of the invention or fragment thereof.

Preferably the kit also contains a bacterial protein-binding moiety, e.g. a second antibody, capable of binding to the bacterial protein or fragment thereof, which may be used to immobilize the bacterium or part thereof. Conveniently the kit also comprises compounds or solutions necessary for the development of an identifiable signal from the signalling means.

Additionally, the kit may also include means for standardization of the assay or for comparative purposes.

Whilst the above assay may be used to assess the levels/presence of bacteria in samples not derived from a patient, e.g. quality control testing of water or food samples or testing for contamination of biological samples, it will be appreciated that a major use of the assay will-be for the purposes of determining the presence of a bacterium or parts thereof in an animal body, which may or may not be associated with disease symptoms. Thus the method may be used to diagnose pathological conditions or to characterize or serotype the type of infection.

Thus, viewed from a further aspect the present invention provides a method of diagnosing infection of a human or non-human animal by a bacterium, wherein said method comprises at least the step of assessing the presence or amount of bacterial proteins of the invention or fragments thereof or nucleic acid molecules encoding said proteins in a sample from said human or non-human animal.

The diagnostic test may be used to determine whether a patient is infected, the extent of infection or to monitor the efficacy of treatment and/or progression of the disease. Patients which are diagnosed may thus be asymptomatic at the time of diagnosis.

Samples which are appropriate for testing will depend on the bacterium and its usual site of infection/location within the body. However, conveniently, body wastes and fluids of the patient, such as urine, faeces, blood, sperm, spinal fluid, saliva, lymph, expectorated matter (pulmonary patients), placenta, biopsy material etc. are used as the sample.

Diagnosis of infection by a bacterium may also be performed in vivo by for example testing for hypersensitivity to said bacterium. This may be determined by superficial, intracutaneous or subcutaneous determination of delayed or immediate hypersensitivity. For example, to determine if delayed type hypersensitivity occurs, animals may be injected intradermally (e.g. at a shaved site along the backbone, flank or peritoneum) with 0.1 to 0.2 ml of the antigen six weeks after initial antigen administration. The antigen may be in purified form or administered as live recombinant bacteria. The results may then be checked within 24-48 hours. Positive reactions are identified by reddening, swelling or necrosis at the site of administration of 5 mm or more in diameter.

Thus viewed from a further aspect the present invention provides a method of diagnosing infection of a human or non-human animal by a bacterium by assessing the reaction of said animal to presentation of the bacterial protein of the invention obtainable from said bacterium. Said presentation may be locally or systemically and the reaction to be assessed may be any reaction normally associated with hypersensitivity, e.g. inflammation, itching etc.

Bacterial glycoprotein polymers of the invention have surprisingly also been found to exhibit anti-proliferative, cytotoxic effects on rapidly growing cells (see Example 2.8). In this respect the bacterial glycoprotein polymers of the invention exhibit similarities to some macroorganism-derived cytokines (e.g. TNF-αor interferon) which play an important role in regulating the immune system and are used in medicine. Recombinant microorganisms have been used to genetically engineer cytokines and immunomodulators, which are generated for the purpose of drug development. The sequences of these cytokines have however no sequence similarity to the tolins described herein.

As illustrated in the Examples, bacterial glycoprotein polymers of the invention were found to prevent cell proliferation of cells in immortal cell lines and ultimately (in 3 to 4 days) cause their death. In light of the fact that tolins demonstrate nuclease activity in vitro and DNA-binding activity when glycosylated, it is believed that in the eukaryotic cell they also destroy chromosomal DNA which is followed by gradual cell death.

The cell-killing effects of tolins are distinct to the effects achieved by toxins. Known toxins generally achieve their effects by blocking one or more enzymatic reactions leading to immediate cell destruction. In contrast, tolins appear to destroy chromosomal DNA without affecting other functions. Once the DNA has been destroyed cells cease to proliferate but do not perish immediately, continuing to generate required material from existing matrices, e.g. RNA. The cell ultimately dies when all internal resources are exhausted, e.g. from 10 hours to 2-3 days. The absence of rapid destruction of cells prevents the usual complications associated with the use of toxins, ie. serious toxic and non-specific inflammatory complications from the mass decay products of the cells.

Furthermore it has been found that bacterial glycoprotein polymers obtained from different bacteria, e.g. gram negative intracellular pathogenic bacteria, such as F. tularensis or M. bovis, exhibit different anti-proliferative effects on different eukaryotic cells. For example, the bacterial glycoprotein polymers of the invention that were tested exhibited cytotoxicity at different concentrations for different cell types. Thus the different tolins showed specificity for different cell types which clearly has applications when using tolins as anti-proliferative agents.

The varying specificity of bacterial glycoprotein polymers of the invention (as evidenced by their ability to be bound by specific bacteriophages to those bacteria) may be due to the differing selectivity of pathogenic intracellular bacteria with respect to human and animal organs and systems. For example, the pathogens of tularaemia, snive (glanders) and melioidosis primarily attack the haematopoietic system, tuberculosis pathogens primarily attack the lungs, the ovaries and the skeletal system, the gonorrhoea pathogen primarily attacks the mucosal epithelia (vagina, conjunctiva), the meningitis pathogen primarily attacks the serous envelope of the brain, the pathogen of typhoid fever primarily attacks the mucous membrane of the gut, etc. If these differences between pathogenic bacteria are due to the specificity of tolins, this provides the basis of the use of particular tolins for treating or preventing different tumours as a consequence of their antiproliferative and cytotoxic effects.

Indeed it has been found that when sensitive laboratory animals were infected with recombinant microorganisms of the invention expressing tolins, the microorganisms exhibited specificity in respect of the inner organs and systems of those animals which correlated to the specificity of the bacteria from which the recombinant microorganisms were produced (see Example 2.9).

Different bacterial protein polymers of the invention may be used to treat different tumours due to their selectivity and specificity with respect to both normal tissue in the area of the tumours and to the type of tumour to be treated. Bacterial protein polymers may thus be tested and selected according to the type of proliferating cells to be inhibited or eliminated.

Thus viewed from a further aspect the present invention provides a method of identifying a bacterial protein polymer of the invention suitable for use as an anti-proliferative, e.g. cell killing agent for a particular cell type, e.g. tumour cell, comprising at least the steps of a) growing said cells in the absence and presence of different bacterial protein polymers of the invention and b) comparing the number of live cells which remain after a time interval and c) identifying the bacterial protein polymer which inhibits cell proliferation to the greatest extent during said time interval.

To avoid damage to surrounding normal tissue, it is clearly advantageous to determine the damage which the bacterial protein polymer of choice is likely to have on this tissue. Thus, parallel assays may be conducted in which cells of the surrounding tissue, or comparable cells, are grown in the presence of the bacterial protein polymers under study. Ideally, the bacterial protein polymer which exhibits the highest ratio of (% of live normal tissue cells remaining: % live fast growing cells, e.g. tumour cells, remaining) has the most desirable properties for reducing the proliferation, preferably eliminating the fast growing cells under investigation. Furthermore, the dose which is suitable may be optimized using the above test.

Conveniently the test may be performed by treating appropriate cells (numbering for example 1×10⁴-1×10⁶ cells) in cell culture for 2-10 day, e.g. for 5 days, at doses in the order of 1 to 10 μg/ml of culture fluid, e.g. 5 to 50 μg/ml.

The above anti-proliferative/cell killing effects may be used to separate normally proliferating and/or non-proliferating cells from fast growing cells in vitro or in vivo. For example, in a lymphocyte blastotransformation reaction (Surcel et al., 1989, Microbial Pathogenesis, 7: p411-419) a mutagen (antigen, bacterial or eukaryotic cell) producing the blastotransformation of a specific cell population is introduced into a test tube or an animal. The introduction of tolins into a reaction of this kind neutralizes the effect of the mutagen by primarily eliminating (killing) rapidly dividing transformed cells. In vitro this effect may be used for example, by using appropriate bacterial protein polymers to control infection of normal cell cultures or explants by rapidly growing cells. Alternatively, fast-growing cells could be eliminated from the body, e.g. from blood to be returned to the patient, e.g. to avoid metastasis, since the required contact time is quite short (in the order of minutes).

The invention thus provides bacterial protein polymers of the invention for use as anti-proliferative agents and use of bacterial protein polymers of the invention to alter the proliferation of cells. In vivo, the bacterial protein polymers have applications for treating any rapidly growing cells, particularly those which are abnormal, e.g. tumours (especially malignancies such as cancer) or leukaemia, in a human or non-human animal.

Thus viewed from a further aspect the present invention provides a method of treating or preventing a condition associated with rapidly growing cells, e.g. a tumour, in a human or non-human animal comprising administering to said animal a bacterial protein polymer of the invention. Alternatively viewed, the present invention provides bacterial protein polymers-of the invention for use as a medicament, particularly for use in treating or preventing conditions associated with rapidly growing cells, e.g. a tumour. Furthermore, the invention provides the use of bacterial protein polymers of the invention for the preparation of a medicament for the treatment or prevention of conditions associated with rapidly growing cells, e.g. tumours.

As used herein, “treating” refers to reducing the rate of proliferation of the rapidly growing cells, e.g. by halting proliferation, causing differentiation or causing some cell death. With respect to tumours, “treating” refers to improving the state of the tumour either by altering the rate of its growth, preferably by preventing its further growth, especially preferably by reducing or eliminating said tumour. With respect to leukaemia, “treatment” refers to normalization of the blood constituents, preferably by reducing the number of, or removing, immature blood cells. “Preventing” said conditions refers to the use of bacterial protein polymers of the invention for prophylaxis, in particular of individuals with a history of, or at risk from, conditions associated with rapidly growing cells, in particular for preventing tumour development, states of leukaemia, or immune reactions or disorders.

As used herein, rapidly growing cells include any cells which exhibit accelerated proliferation relative to cells of a similar type, e.g. surrounding tissue or haematopoietic system. In particular the invention is directed to treating or preventing abnormally rapidly growing cells, ie. those not normally observed in comparable normal individuals. The rapidly growing cells may result through disease or may be the body's reaction to a particular event, e.g introduction of foreign material into said body. The body's natural immune reaction to infection by undesirable entities is not considered to constitute an abnormal growth of cells (immune cells), despite the lack of a comparable event in uninfected but otherwise comparable individuals.

In particular tumour growth and leukaemia constitute such abnormal growth. Furthermore, bacterial protein polymers of the invention may be used to treat or prevent activated immune responses, e.g. in autoimmune diseases, or to prevent rejection in transplantation surgery. Thus, the bacterial protein polymers of the invention may be used as immunosuppression agents. Tolins may be effective, for example, in polycythaemia vera and spurious polycythaemia, in which excessive proliferation of all cellular components of the blood is observed.

As mentioned above tumours which may be treated may be cancerous, e.g. carcinomas, sarcomas, glioma, melanoma and Hodgkin's disease, including cancers of the breast, gut, prostate, lung and ovary. Alternatively, the tumour to be treated may be benign, for example papillomatosis and fibromatosis.

As described above, the strategy for selecting tolins to treat abnormally growing cells may be determined by pathomorphological manifestations, principally lesions of particular organs (systems) due to pathogenic intracellular bacteria. For example, tolins isolated from the pathogen of dysentery or typhoid fever are preferentially used to treat malignant and benign tumours of the gut, while tolins isolated from the pathogens of tuberculosis, meningitis and tularaemia, snive (glanders) and melioidosis are preferentially used to treat lung and ovarian cancer, cerebral cancer and various leukaemias, respectively. The last class of tolins may also be used as immunosuppressants. In determining appropriate doses for treating abnormally growing cells in vivo, as mentioned above, it is necessary to determine appropriate dosages at which maximal cytotoxic effect is achieved on the undesired cells, with minimal adverse effects on surrounding normal tissue. Adverse effects may be minimized by local administration to the affected area.

Tolins are taken up into the cytoplasm of tumour cells, which may occur through association with a receptor on those cells, where they destroy the chromosomal DNA of the cell, prevent proliferation and cause cell death within a few days. If appropriately-labelled, they may therefore be used as markers for fast-growing cells, for example in the diagnosis of tumours. Depending on the portion of the tolins which bind to the cells (the acceptor region), it will be appreciated that tolin fragments (e.g. an unglycosylated polymer or a monomer) may be sufficient for use as a marker.

The present invention thus further provides a method of diagnosing the presence or location of fast-growing cells, e.g. tumour cells, in a human or non-human animal, wherein said method comprises at least the step of assessing the association of bacterial proteins of the invention with cells of said animal.

As used herein, “association” refers to binding to receptors (where these are present) on the surface of eukaryotic cells, or internalization within the cells.

Bacterial protein polymers of the invention for use in the above described clinical methods include functionally-equivalent variants, derivatives, fragments and precursors thereof, which particularly include pharmaceutically acceptable salts thereof. Pharmaceutically acceptable salts may be readily prepared using counterions and techniques well known in the art.

The invention further extends to pharmaceutical compositions comprising one or more bacterial protein polymers of the invention, together with at least one pharmaceutically acceptable carrier, diluent or excipient, and their use in treating or preventing the above described conditions.

It will be appreciated that the following discussion relating to pharmaceutical compositions of the invention applies with respect to suitable excipients etc. and formulations of the compositions also to the vaccine compositions described herein.

The active ingredient in such compositions may comprise from about 0.01% to about 99% by weight of the formulation, preferably from about 0.1 to about 50%, for example 10%. By “pharmaceutically acceptable” is meant that the ingredient must be compatible with other ingredients of the compositions as well as physiologically acceptable to the recipient.

Pharmaceutical compositions according to the invention may be formulated in conventional manner using readily available ingredients. Thus, the active ingredient may be incorporated, optionally together with other active substances, with one or more conventional carriers, diluents and/or excipients, to produce conventional galenic preparations such as tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, sterile packaged powders, and the like.

Examples of suitable carriers, excipients, and diluents are lactose, dextrose, sucrose, sorbitol, mannitol, starches, gum acacia, calcium phosphate, aglinates, tragacanth, gelatin, calcium silicate, microcrystalline cellulose, polyvinylpyrrolidone, cellulose, water syrup, water, water/ethanol, water/glycol, water/polyethylene glycol, propylene glycol, methyl cellulose, methylhydroxybenzoates, propyl hydroxybenzoates, talc, magnesium stearate, mineral oil or fatty substances such as hard fat or suitable mixtures thereof. The compositions may additionally include lubricating agents, wetting agents, emulsifying agents, suspending agents, preserving agents, sweetening agents, flavouring agents, and the like. The compositions of the invention may be formulated so as to provide quick, sustained or delayed release of the active ingredient after administration to the patient by employing procedures well known in the art.

The compositions for the treatment or prophylaxis of oncological diseases and abnormal states are preferably formulated in a unit dosage form, e.g. with each dosage containing from about 0.01 mg to about 1 g of the active ingredient, e.g. 0.05 mg to 0.5 g, for a human e.g. 1-100 mg. Formulations for vaccination providing the same dose as for the pharmaceutical applications are preferably prepared in multidose form (from 3-5 to 20 doses for humans and 5-10 to 50 doses for animals). The above doses apply to administration of the purified bacterial proteins, or administration of a live vaccine (e.g. recombinant microorganism).

The precise dosage of the active compound to be administered and the length of the course of treatment will, of course, depend on a number of factors including for example, the age and weight of the patient, the specific condition requiring treatment and its severity, and the route of administration. Generally however, an effective dose may lie in the range of from about 1 μg/kg to about 10 mg/kg, e.g. from about 1 mg to 0.2 g per day, depending on the animal to be treated, taken as a single dose. Thus for example, an appropriate daily dose for an adult, may be from 0.5 mg to 0.5 g per day, e.g. 1 to 100 mg per day.

The administration may be systemic or topical and may be by any suitable method known in the medicinal arts, as mentioned previously in respect of administration of vaccine compositions.

As mentioned previously, bacterial glycoprotein polymers of the invention have furthermore been found to have non-specific DNA and RNA nuclease activity which is operative in vitro. The present invention thus provides the use of bacterial protein polymers of the invention to cleave nucleic acid molecules in vitro, for example in crude DNA preparations, or alternatively viewed, provides a method of cleaving nucleic acid molecules in vitro, wherein said nucleic acid material is contacted with bacterial protein polymers of the invention for a time and a concentration appropriate to result in partial or complete cleavage of said nucleic acid molecules.

The invention will now be described in more detail by way of the following non-limiting Examples, in which

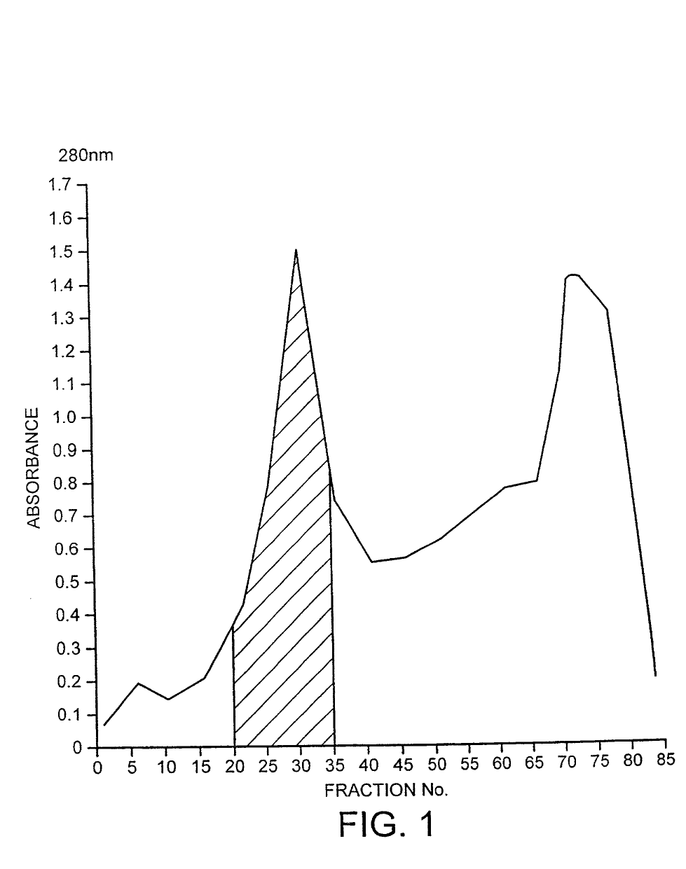

FIG. 1 shows gel filtration separation of tolin-enriched lysate proteins of RB7;

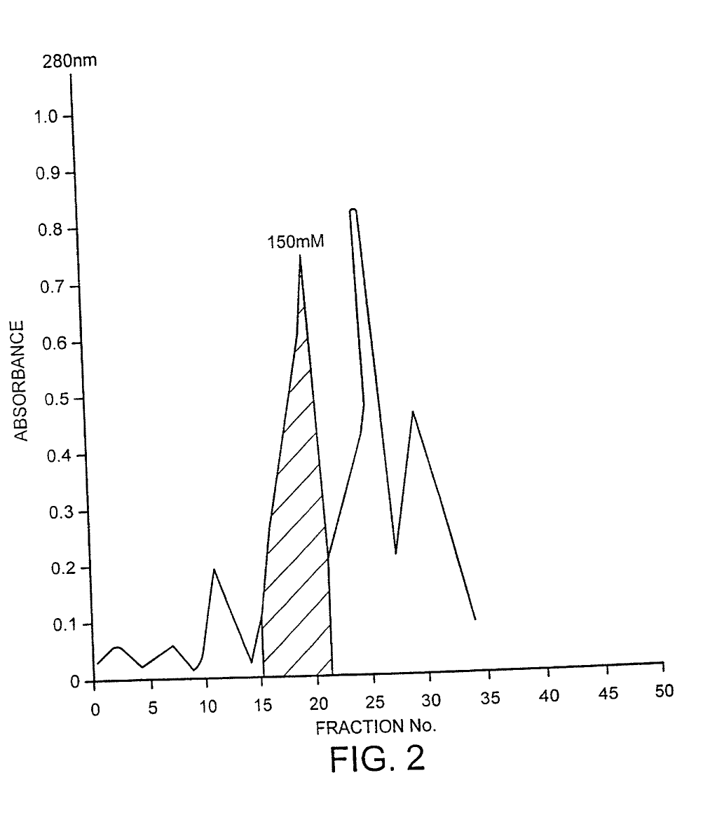

FIG. 2 shows ion-exchange chromatography of the tolin-containing fractions derived from FIG. 1;

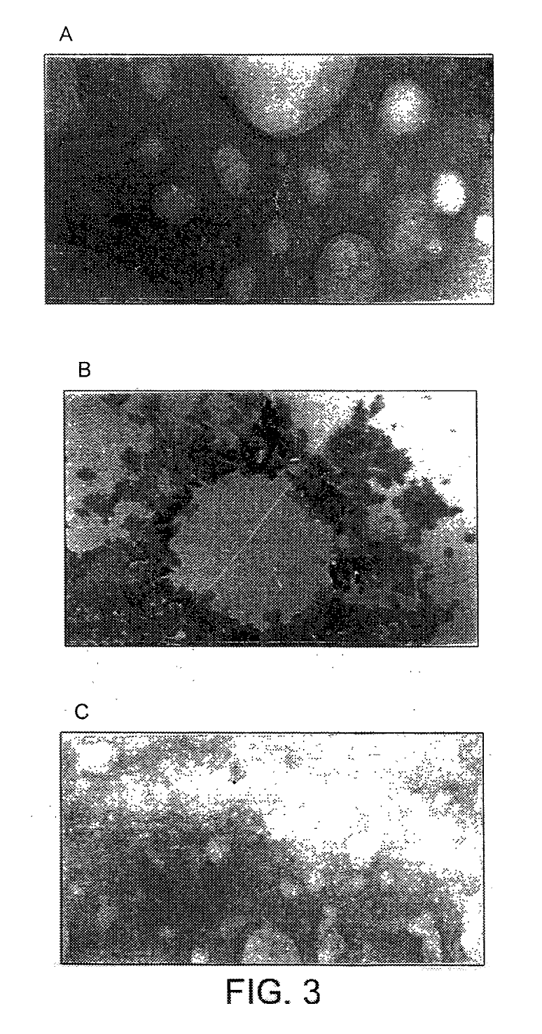

FIG. 3 shows non-specific aggregation of low level glycosylated tolins on changing pH from 2.0 to 7.5 as evidenced by electron microscopy contrasted with 2% uranyl acetate solution, scale ×50,000;

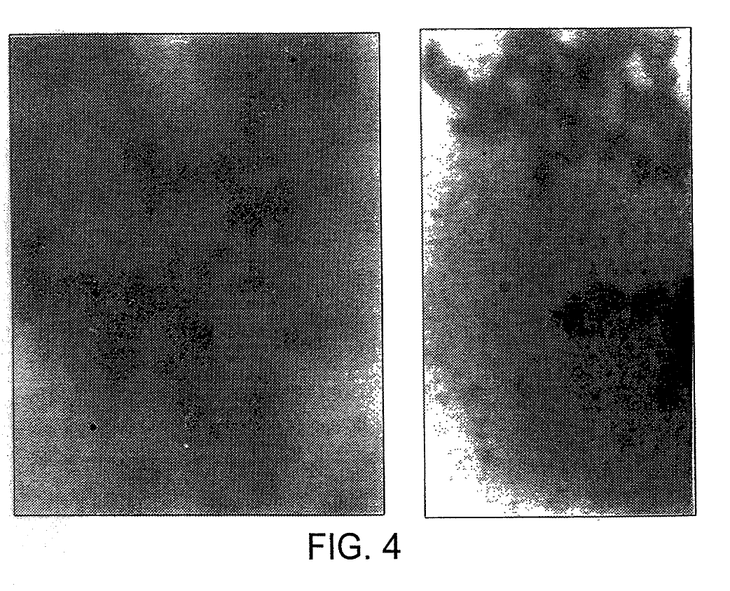

FIG. 4 shows a purified tolin preparation from the R-form of Francisella tularensis in EM contrast with uranyl acetate (scale ×50,000);

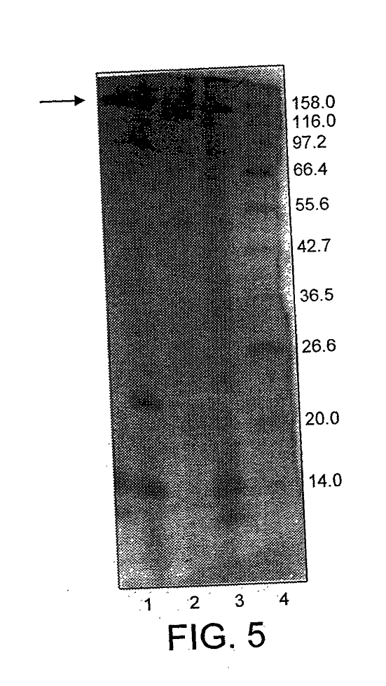

FIG. 5 shows a 15% PAGE non-denaturing gel of various purified tolin preparations in which the tolin in polymeric form is indicated by the arrow; lane 1: tolin from the R-form of F. tularensis, isolated by gel filtration and ion-exchange chromatography, lane 2: tolin from the vaccine strain 15 NIIEG, isolated by gel filtration and ion-exchange chromatography, lane 3: tolin from the vaccine strain 15NIIEG, isolated by HPLC, lane 4: marker lane;

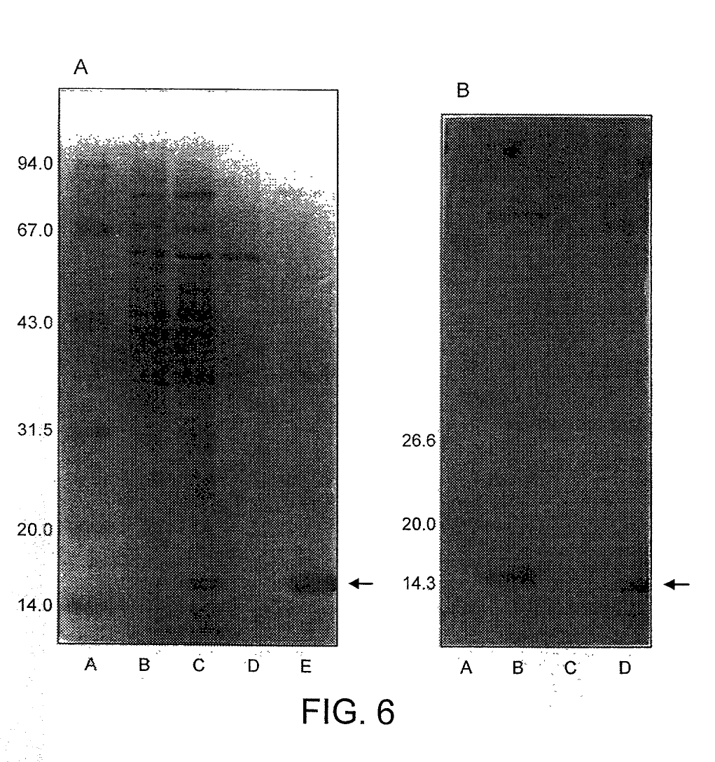

FIG. 6 shows 15% SDS-PAGE non-denaturing gels with tolins present in lysates or in purified form; A, lane a: protein markers; lane b,c—cell lysate of R-form Francisella tularensis; lane d—individual protein lysate of R-form F. tularensis lysate; lane e—purified tolin of R-form F. tularensis, B. lane a: protein markers; lane b—purified tolin of R-form F. tularensis; lane d—purified tolin of vaccine strain F. tularensis;

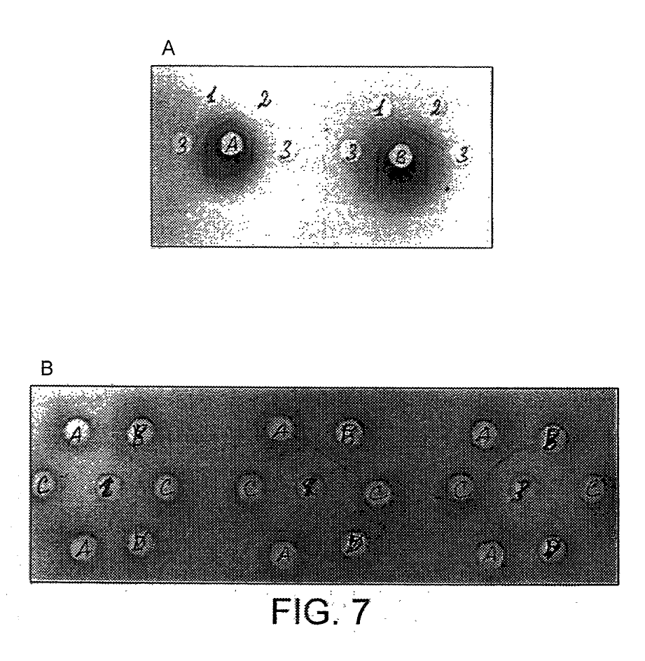

FIG. 7 shows tolin interaction with specific rabbit serum in diffuse precipitation reaction (DPR); I, A—specific serum against RB26, B—specific serum against RB7, 1—tolin from R-form F. tularensis, 2—tolin from vaccine strain 15 NIIEG, 3—tolin from RB26; II, A—specific serum to RB26, B—specific serum to RB7, C—specific serum to RM28, D—specific serum to R-strain, 1—tolin from R-form, 2—tolin from vaccine strain 15 NIIEG, 3—tolin from RB26;

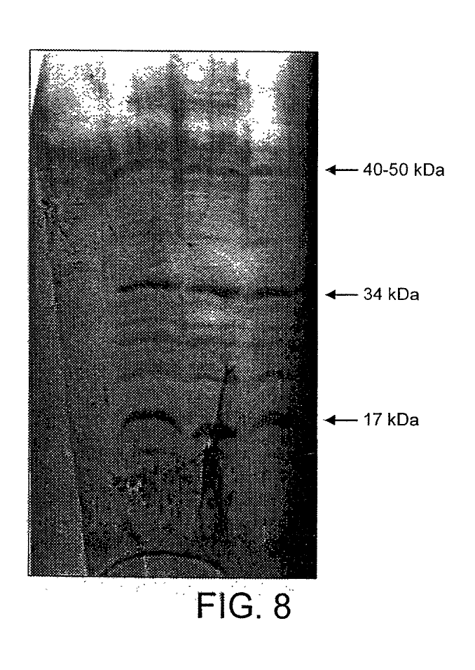

FIG. 8 shows an immunoblot of the lysate of vaccine strain 15 NIIEG probed with normal rabbit serum antibodies. Binding to the monomeric, dimeric and trimeric forms is shown with the arrows;

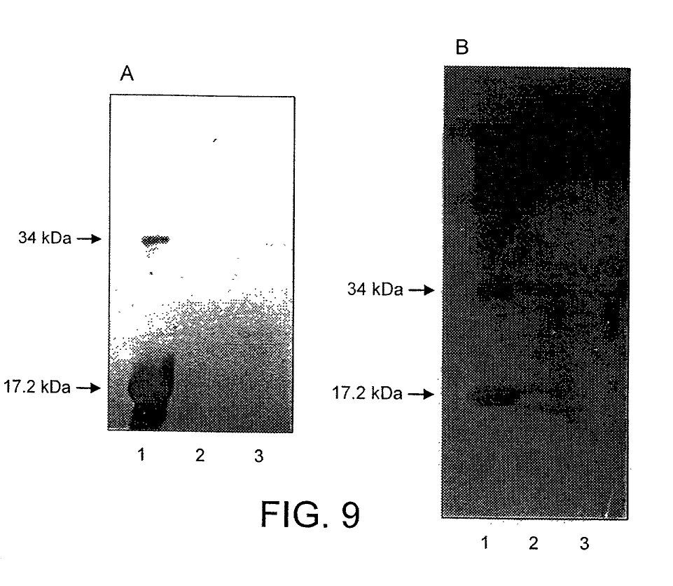

FIG. 9 shows an immunoblot of purified tolins probed with TNF-alpha antibodies, A—antibody 1, B—antibody 2 (obtained from Research Institute of Gematology, the Laboratory of Cell and Molecular Immunology, Dolginovsky Trakt 160, 223059, Minsk, Belorussia (product name: Test-system for the determination of TNF-α)), lane 1 —TNF-alpha (as control), lane 2—tolin from R-form of F. tularensis, lane 3 —tolin from vaccine stain 15 NIIEG;

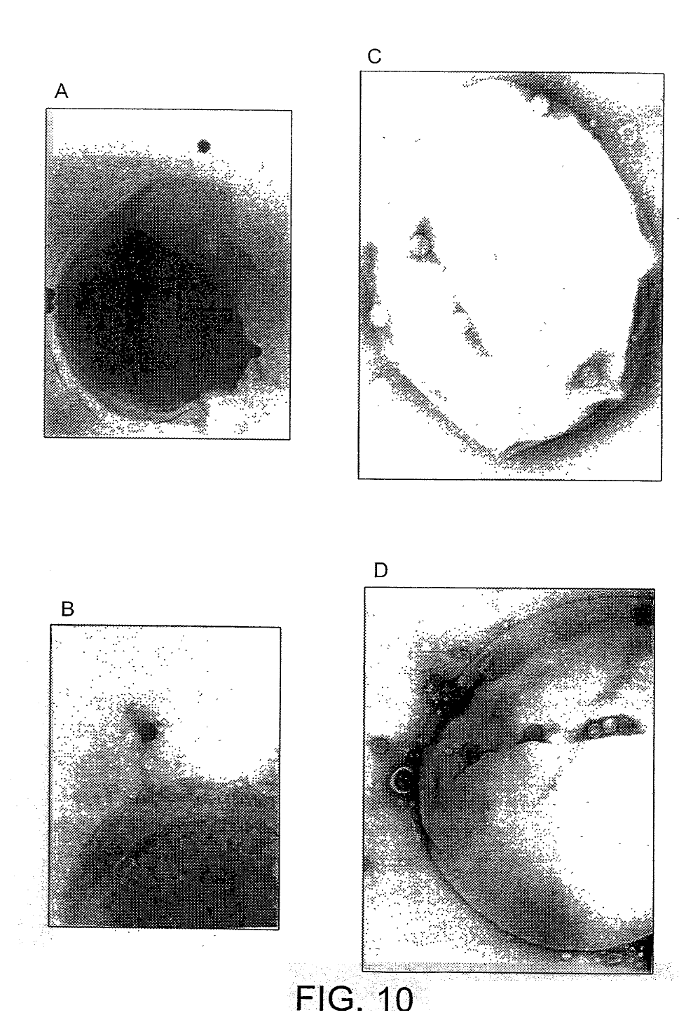

FIG. 10 shows the binding of the tuberculosis bacteriophage MTPH2 to the capsule (A,B) and external membrane (C,D) of F. tularensis cells containing tolins of tuberculosis origin, examined by electron microscopy contrasted with uranyl acetate, scale ×50,000;

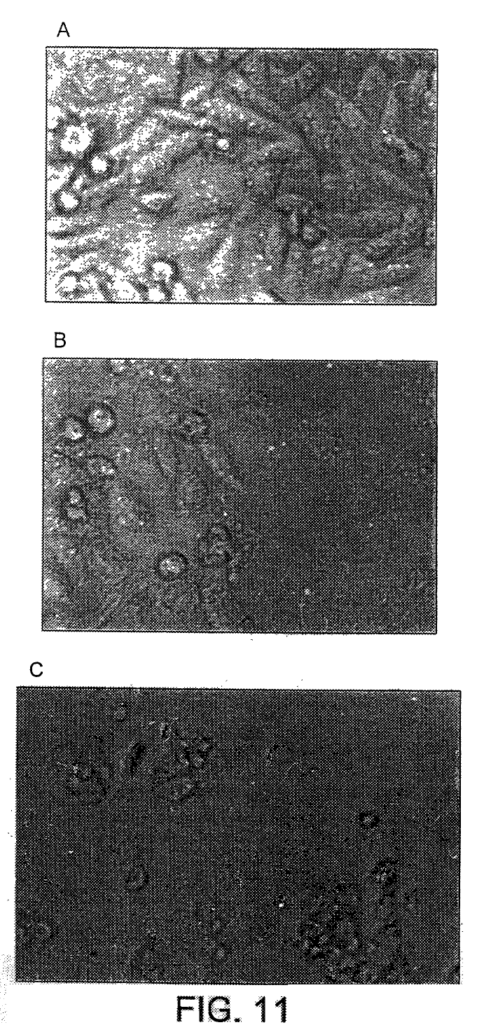

FIG. 11 shows the antiproliferative and cytotoxic effects of tolins on CHO cells, A—normal cells, B—cells treated with tolin purified from RB26 (protein concentration 5.0 μg/ml) showing an antiproliferative effect, C—cells treated with tolin purified from RE26 (protein concentration 10 μg/ml) showing a cytotoxic effect.

### EXAMPLE 1

### ISOLATION OF BACTERIAL GLYCOPROTEIN POLYMERS OF THE INVENTION

1.1 General Methodology

Isolation is performed as follows:

1. Wash a 3-day culture of bacterial cells from solid nutrient media (60 petri dishes) using deionized chilled water (8 ml water/petri dish).

2. Pool the cells (concentration of suspension 10¹¹-10¹² cells/ml). (The biomass may be frozen and stored at -55° C.)

3. Disrupt the cell suspension 3-5 times, e.g. by sonication, for 1 minute each time in the cold (4-8° C.).

4. Clarify the lysate by centrifugation at 7000 rpm for 50 minutes at 4-8° C. (Protein concentration in the lysate between 2 and 5 mg/ml.)

5. Precipitate lysate proteins over 16-18 h at 8° C. by adding dry (NH₄)₂SO₄ until 50% saturation is achieved.

6. Separate precipitated proteins by centrifuging the lysate at 7000 rpm for 50 minutes at 4-8° C. Tolins concentrate in the supernatant.

7. Precipitate proteins in the supernatant over 16-18 hours at 4-8° C. by adding dry (NH₄)₂SO₄ until 100% saturation is achieved.

8. Separate the precipitated proteins by centrifuging the supernatant at 7000 rpm for 50 minutes at 4-8° C.

9. Resuspend the precipitate in 20-25 ml 10 mM Tris buffer, pH 7.5 at 8° C.

10. Dialyse the suspension against a 100-fold volume of 10 mM Tris buffer, pH 7.5, over 16-18 hours (replacing the buffer once) at 8° C. (protein concentration between 5 and 10 mg/ml).

11. Dialyse the suspension against a 100-fold volume of 50 mM Tris buffer, 100 mM NaCl, pH 7.5 over 16-18 hours at 8° C., equilibrating a Sephadex G-200 with the same buffer.

12. Filter the specimen through the gel over 20-22 hours at ambient temperature.

13. Dialyse the tolin-containing fraction against 10 mM Tris buffer, pH 7.5, over 16-18 hours at 8° C., equilibrating a DEAE cellulose column with the same buffer.

14. Subject the specimen to ion-exchange chromatography on the column over 2 to 3 hours at ambient temperature with a stepped gradient of NaCl, 10 mM Tris, pH 7.5.

The concentration and activity of the tolin is checked at stages 4, 6, 10, 13 and 14 above.

Using the above method, the fractions which are obtained which contain the tolins have a purity of between 60 and 75% (of total protein) and may be used experimentally.

1.2 Experiments performed using the above general methodology

1.2.1 Purification from RB7

Gel filtration was performed on Sephadex G200 (84×2.6 cm) in 50 mM Tris pH 7.5, 100 mM NaCl using 24 ml of a RB7 preparation (after step 10 above) with a protein concentration of 2.7 mg/ml. The results are shown in FIG. 1. Tolin-enriched material appeared in fractions 20-35.

The above fractions (21 ml, 0.7 mg/ml) were then applied to a DEAE cellulose column (5×1.6 cm) in 10 mM Tris pH 7.5 and eluted step-wise with 50, 100, 150, 250 and 500 mM NaCl. The results are shown in FIG. 2. The tolin-containing fractions are hatched.

The purity of the tolin and preservation of the polymeric form was established by SDS-PAGE electrophoresis and protein concentration determined by the Lowry method.

1.2.2 Alternative purification technique

Tolin-enriched fractions (after step 10 above) were subjected to gel filtration as described below.

The quantity of protein in the samples was estimated using the analytical HPLC-system on a C₁₈ column (4×150 mm I.D.). Analysis of the samples which were obtained was carried out by reverse phase HPLC using a Gilson Model (France) liquid chromatography apparatus. The column (4.5×250 mm I.D., stainless steel) was packed with Nucleosil-C₁₈ with a particle size of 7 μm and pore size of 100 Å (“Biotronik”, Germany). The samples were subject to a linear gradient of 0 to 70% water/acetonitrile in 0.15 % TFA (by volume). Spectrophotometric detection was at 220 nm. The flow rate of the eluent was iml/min. For the analytical separations the volume of the samples which were injected was 100 μl and in the micropreparative separations was 1.5-2.0 μl depending on protein concentration.

In the first stage of purification, a Diacard C₈/t column (16×250 mm) was used. Its characteristics were: bonded octylsilane-phase, 6 μm particle size, 130 Å pore size. The separation was carried out in a gradient of acetonitrile/water with the addition of 0.1% TFA. The flow rate of the eluent was 5 ml/min. The collected fractions were analysed for the desired protein. Acetonitrile was then evaporated. The purity of the tolin and preservation of its polymeric form were estimated by the method of SDS-PAGE electrophoresis in BNB buffer using the method of Laemmli. Protein content was determined by the Lowry method.

For the final purification column (4.6×250 mm I.D., stainless steel) packed with Nucleosil-C₁₈ with a particle size of 7 μm and a pore size of 100 Å was used. The mobile phase and gradient were the same as in the first stage of purification. Detection was at 220 nm. Tolin eluted in single fractions at 51-52% acetonitrile. Analysis of the collected fractions was carried out using the method of SDS-PAGE electrophoresis. The quality of the obtained protein was confirmed in a gradient of 30 to 70% acetonitrile over 40 mins.

The tolin purity and preservation of the polymeric form was determined as mentioned above.

The above method resulted in tolins with a purity of 95% (relative to total protein). Although this method is used to obtain tolins (with a polysaccharide content of between 0.1 and 1.0%, which is sufficient for biological activity), the purification conditions (pH 2.0 to 3.0), result in proteins which are not at their pH optimum (optimum biological, immunological and enzymatic activity of tolins is exhibited at pH 7.4-7.5). To obtain tolins in active form, the solution was neutralized. Precipitation of as much as 90% of the protein occurs (see FIG. 3). The remaining approximately 10% which is enzymatically, antigenically and biologically active is shown in FIG. 4.

1.2.3 Isolation of unglycosylated monomers

The glycoprotein polymer was obtained from the R-form of F. tularensis by purification as described in Example 1.1, after step 10. Thereafter further purification was conducted as described in Example 1.2.1. (Alternative techniques may also be used, particularly those which result in minimum levels of polysaccharide in the purified preparation.)

The preparation was then heated to 100° C. in BNB-buffer and the sample was applied to a 15% SDS-PAGE gel (50 V, 14 hours). The gel strip corresponding to 17 kDa was removed, ground in a mortar and placed in a buffer of 10 mM tris-HCl, 150 mM NaCl, pH 7.5. The monomers were eluted from the gel in a sealed vessel with shaking for 12 hours at 8-10° C.

The resulting eluate was purified to remove SDS and polyacrylamide contaminants by dialysis against a 100-fold volume of the same buffer.

This protein monomer solution was concentrated to 1/10 th of the initial volume using polyethylene glycol with a molecular weight of 40 kDa.

The resulting tolin protein monomer was found to be chromatographically pure without polysaccharide. A polymer form was also identified (see Example 2.10).

### EXAMPLE 2 CHARACTERIZATION OF BACTERIAL GLYCOPROTEIN POLYMERS (TOLINS) OF THE INVENTION

2.1 Molecular weight determination

An illustrative PAGE non-denaturing gel of various purified tolins (according to the method described in Example 1) is shown in FIG. 5. Tolins can be observed in the region of 116 to 158 kDa. FIG. 6 shows separation on a 15% SDS-PAGE denaturing gel showing the lysate from which tolins are purified and the purified tolin which runs as a band of approximately 17 kDa under the denaturing conditions.

2.2 Carbohydrate analysis

Hydrolytic cleavage of the glycoprotein: Protein sample (0.1 to 0.5 mg obtained after step 4 of Example 1 =bacterial lysate) from R-form F. tularensis, the vaccine strain 15 NIIEG and the recombinant strain RVT-1 was heated in 1.0 N sulfuric acid (5 hours 101° C.), or, for the purified protein, was dissolved in 1 ml of 1.1N HCl and heated for 5 hours at 101° C. The resulting mixture was evaporated to dryness (SpeedVac). Ambiguous cases demanded parallel experiments with more severe treatment conditions (2 N HCl, 5h).

Chemical derivatization of “usual” carbohydrates (aldoses, ketoses): The dried residue was treated with 100 ml of 2% pyridinic HONH₂.HCl in order to convert carbonyl groups into oxime moieties (30 min, 75° C.). Then 1 ml of sylilating mixture was added (trimethylchlorosilane, hexamethyldisilazane and pyridine, 1:3:9) and the sample was heated for 40 minutes (75° C.). The resulting solution of carbohydrate oxime per-TMS-ethers was analysed. The solvent, water, was analyzed as a control.

Chemical derivatization of amino sugars: The dried hydrolysate was treated with 500 ml of “strong” silanizing mixture consisting of bis(trimethylsilyl)acetamide, trimethylchlorosilane and acetonitrile (100:1:400), 15 minutes, 75° C. Substitution of active hydrogens in all 6 positions takes place thus preventing peak tailing and broadening.

Gas chromatographic analysis: The following conditions were used:—Column of fused silica, 30 m×0.53 mm, stationary phase —immobilized methylphenylsilicone HP-5 on a capillary gas chromatograph HP5890 (Hewlett-Packard), detection FID, 295° C. Injection was performed cold onto the column with 2 ml. Temperature programming—a) from 100°C. (1 min) to 285° C. (10 min), 6° C./min (for aldoses and ketoses); b) from 150° C. (1 min) to 285°(10 min), 7° C./min (for aminosugars). Data was examined using integrator HP3396A. Calibration was performed with standard solutions with 1.0 to 7.0 mg/ml of each sugar. The experimental detection limits were 0.3 to 0.5 mg of each monosaccharide (corresponding to 0.1 to 1.0% w/w content of each monosaccharide in the starting glycoprotein). The carbohydrate compositions of the bacterial lysates and the purified tolins were examined.

Results

The specimens from the bacterial lysates were virtually identical for all parameters and contained a considerable amount of glucose (≧80%). Xylose, ribose, rhamnose and glucosamine were also detected. Small admixtures (5-7% each) of some kind of deoxyaldohexose and ketoglucose are theoretically possible. This could only be established more reliably by examining the purified polysaccharide fraction.

In the purified glycoprotein, the presence of the monosaccharides glucose, xylose, rhamnose and ribose were detected. The monosaccharide derivatives glucosamine and galactosamine were absent.

2.3 Lipid Analysis

Chroma tographic investigation

Samples of clarified lysates (produced in accordance with step 4, Example 1) of the R-form of F. tularensis, the vaccine strain 15 NIIEG and the recombinant strain RVT-1, in addition to purified tolins, were examined for the presence of aliphatic acids in the interval C₁₆₋₁₈ or C₁₄₋₂₀. Samples were dried and saponified in 0.5 N NaOH in methanol, methylated with 2% H₂SO₄ in methanol according to known techniques, dissolved in water and the ester extracted with 0.5 ml analytical grade hexane. For identification of the lipids the gas chromatography apparatus HP5890 was used. The column which was used was fused silica (30 m×0.53 mm), the stationary phase was immobilized methylphenylsilicone HP-5.2 ml samples were injected cold onto the column. The run was performed at 240° C. Calibration was performed with 3.5 mg of palmitic acid and 1.7 mg of stearic acid. Control blank runs were also performed.

Results C₁₆₋₁₈ were present in all lysate samples, but at negligible levels compared to the levels of proteins and polysaccharides. In the tolins, fatty acids C₁₄-C₂₀ were found to be absent from tolins.

Chloroform treatment

Lysates and supernatants enriched with tolins (produced in accordance with steps 4 and 11 of Example 1) were investigated. Chloroform was added into the samples (1:10) at room temperature and carefully mixed to obtain a homogeneous opaque solution. The resulting mixture was centrifuged at 5000 rpm for 15 minutes. The supernatant was removed. This was repeated twice. The lipid-free samples containing tolins were kept overnight at 4° C. to allow complete evaporation of chloroform. The capacity of these tolins to react with specific serum, their nuclease activity, and the presence of the polymeric etc. form was then investigated by immunoelectrophoresis and protein electrophoresis and methods described herein.

Results

On treatment with chloroform the polymeric form and immunological, nuclease and biological activities of tolins are not lost.

2.4 Antiaenicity

Interaction with rabbit serum prepared to particular tolins Preparation of tolin-specific antiserum:

Tolins produced in accordance with step 14 of Example 1 were used as antigen. Antiserum was prepared in rabbits with a body weight of 3-4 kg. A first immunisation was performed by hypodermic administration in the area of the rear limb lymph node. lml of the tolin preparation was injected (about 160 μg) in buffer solution with 100 mM NaCl and 10 mM Tris-HCl at pH 7.5 mixed with 1ml of incomplete Freuds adjuvant. Seven days later a second immunisation of the animal was performed with the same mixture by intramuscular injection at the same size. Seven days later a third immunisation was performed by intramuscular injection of the same preparation in the area of the other rear limb lymph node. Seven days later a fourth immunisation was performed in the area of the rear limb lymph node with a 2 ml solution containing tolin in the above mentioned buffer solution but without adjuvant. Seven days later 30-50 ml of blood sample was removed from the rabbit's ear vein. The serum was prepared by stirring the blood with a glass rod and incubating for 20 minutes at 37° C. and further for 14-16 hours at 8° C. to enhance full layer separation. The serum was removed with a dropper, centrifuged for 10 minutes at 3000 rpm. The supernatant was stored at -20° C. The presence of specific antibodies in this serum was validated with Ochterlony immunodiffusion analysis.

Ochterlony—Experimental procedures and assaying of serological responses were performed routinely.

Results

The serum exhibited specific reactions with tolins in the course of diffuse precipitation reactions in the range of 1:16-1:32. FIG. 7 shows the specificity and cross-reaction of serum prepared by challenge with different tolins. It will be observed that some cross-reactivity occurs.

Interaction of normal rabbit serum antibodies with tolins in the monomeric, dimeric and trimeric states

Cell lysates of the vaccine strain of 15 NIIEG containing tolins were probed in immunoblots with normal rabbit serum antibodies. The results are shown in FIG. 8 in which binding of normal rabbit serum antibodies to tolin monomers, dimers and trimers can be observed.

Interaction of tolins with TNF-alpha antibodies

Purified tolins were immunoblotted with TNF-alpha antibodies obtained from 2 different sources. [See legend to FIG. 9 above] The results are shown in FIG. 9 in which it will be observed that TNF-alpha antibodies bind to the monomeric and dimeric form of the tolins.

2.5 Nuclease activity

A. Medium saline restriction buffer (×10) was added to the amount of 1/10 of the volume of the reaction mixture with a 5 μl solution containing 0.8 μg protein (tolin) and 10 μl solution containing 0.8 μg chromosomal DNA of Mycobacterium bovis (BCG). The temperature was maintained at 37° C. in a water bath for 90 minutes. Degradation of chromosomal DNA was >50%. Native DNA was partially present.

B. Medium saline restriction buffer (×10) was added to the amount of 1/10 of the volume of the reaction mixture with a 10 μl solution containing 1.6 μg protein (tolin) and 10 μl solution containing 1.2 μg chromosomal DNA of Mycobacterium bovis (BCG). The temperature was maintained at 37° C. in a water bath for 60 minutes. Native chromosomal DNA was absent. A “tail” of chromosomal DNA fragments was observed in the middle section of the gel. The findings were analysed by electrophoresis in a 0.7% agarose gel. As a control the same amount of native chromosomal DNA of M. bovis (BCG) in a similar buffer was used. Protein concentration was determined according to Bradford. (Restriction buffer: 50 mM NaCl, 10 mM MgCl₂, 10 mM Tris-HCl, pH 7.5, 1 mM dithiothreitol.)

C. RNAse activity was determined by the method described above using 10 μl of a eukaryotic tRNA solution at a concentration of 1.5 to 2.0 μg/μl.

2.6 Specific binding of bacteriophages

Recombinant cells of F. tularensis (RB7, RB26) containing fragments of M. bovis (BCG) DNA expressing the TB tolin were washed in distilled water with salt buffer containing low Mg²⁺(10 mM tris-HCl, 1-mM MgCl₂, pH 7.0) to allow phage binding. MTPH2 (DS6A) Phage was then added in a cell:bacteriophage ratio of 1:30-50. This mixture was incubated for 20 minutes at 37° C. Phage binding was then examined according to known techniques by electron microscopy. The results are shown in FIG. 10.

2.7 Sequencing

The 17 kDa protein derived from HPLC, with purity confirmed by denaturing SDS-PAGE and mass spectrometry, of purified preparations from the R-form, RB7-I tol, 15 R tol and R32 tol were sequenced. (The same results were obtained for the first 45 amino acids using monomers isolated by SDS-PAGE.) 2 partial sequences were identified which are described in the text. Amino acid sequencing was performed by standard techniques (see in this connection Omtvedt et al., 1997, Scand. J. Immunol., 45, p551-556). The full sequence of the 17 kDa protein was determined after trypsin fragmentation followed by cleavage with endoproteinase and CNBr. The method is described more fully below.

Methods

Protein purification—The 17 kDa protein was purified by RP-HPLC on an Aquapore RP-300 column (10×200 mm) with a linear gradient of acetonitrile from 10-50% over 60 minutes, at a flow rate of iml/min. Protein was detected on a spectrophotometer at 214 nm and vessel width of 10 mm. The protein fraction was dried in a SpeedVac. Gel electrophoresis—SDS-PAGE on a 12.5% gel was performed on a mini-protein cell (Bio-Rad) according to the method of Laemmli.

Determination of cysteine residue(s)—The number or presence of cysteine residues in the 17 kDa protein was determined by mass difference of the protein by mass spectrometry before and after alkylation with 4-vinylpyridine.

Amino acid analysis—Protein samples (two) were vapour hydrolyzed with trifluoroacetic acid and analyzed on a Hitachi amino acid analyzer, model 835 (ninhydrin method). N-terminal sequence analysis—Automatic N-terminal sequence analysis was performed on a model 810 Kanuer protein/peptide sequencer equipped with a model 120 A PTX Analyzer (Perkin-Elmer/Applied Biosystems).

Mass spectrometry of protein and peptides—Nano electrospray (nano ESI-MS) of the protein was obtained on a Q-TOF mass spectrometer (Micromass Ltd, UK) equipped with a nanoelectrospray ion source. Mass spectra of peptides were obtained on a model Voyager MALDI-TOF mass spectrometer (PerSeptive Biosystems).

Digestion with trypsin—Approximately 400 μg of protein was digested with 4 μg of trypsin in 0.1 M Tris buffer, pH 8 for 4 hours at 37° C. Peptides were separated by RP-HPLC on Aquapore RP-300 (4.6×220 mm) with an acetonitrile gradient from 0-50% over 150 minutes at a flow rate of 0.7 ml/min at 50° C. Peptides were detected at 210nm with a Rapid Spectral Detector (LKB).

Digestion with endoproteinase Glu-C—Approximately 200 μg of protein was digested with 10 μg of endoproteinase Glu-C in 0.1 mM bicarbonate buffer pH 7.8 for 16 hours at room temperature. Peptides were separated as indicated above but with an acetonitrile gradient from 0-50% over 120 minutes at a flow rate of 0.7 ml/min at 50° C.

Cleavage with CNBr—Approximately 300 μg of protein was cleaved in 80% of TFA with 3 μl of 5 M CNBr in acetonitrile for 18 hours at room temperature. The reaction mixture was evaporated to dryness on a Speedvac (Savant) and redissolved in 0.1 M Tris in 5M Gnd-HCl, pH 7.5. Peptides were separated as indicated above but with an acetronitrile gradient of from 5-50% over 90 minutes at a flow rate of 0.7 ml/ml at 50° C.

Results

The 17 kDa protein was found to have the following sequence, consisting of 145 amino acids:

Met Glu Leu Lys Leu Glu Asn Lys Gln Glu Ile Ile Asp Gln Leu

Asn Lys Ile Leu Glu Leu Glu Met Ser Gly Ile Val Arg Tyr Thr

His Tyr Ser Leu Met Ile Ile Gly His Asn Arg Ile Pro Ile Val

Trp Ser Met Gln Ser Gln Ala Ser Glu Ser Leu Thr His Ala Thr

Ala Ala Gly Glu Met Ile Thr His Phe Gly Glu His Pro Ser Leu

Lys Ile Ala Asp Leu Asn Glu Thr Tyr Gln His Asn Ile Asn Asp

Ile Leu Ile Glu Ser Leu Glu His Glu Lys Lys Ala Val Ser Ala

Tyr Tyr Glu Leu Leu Lys Leu Val Asn Gly Lys Ser Ile Ile Leu

Glu Glu Tyr Ala Arg Lys Leu Ile Val Glu Glu Glu Thr His Ile

Gly Glu Val Glu Lys Met Leu Arg Lys Tyr

It will be noted that no cysteine residues were identified. The calculated molecular mass from this sequence is 16804.38Da which correlates well with the observed 16810.27Da. No post-translational modifications were identified.

2.8 Antiproliferative action of tolins

To evaluate cytotoxic activity tolin preparations were dissolved in culture medium (RPMI 1640, Sigma, with 10% FCS and glutamine buffered with 0.05% HEPES Na) to obtain a protein concentration of 10Oyg/ml. This preparation was sterilized by microfiltration through Bio-Rad filters. CHO cells and cells of the myeloma strain P3X63-Ag8.653 (Mα) were used to test cytotoxic activity. Mα cells do not produce immunoglobulins and grow in suspended culture. They have significant potential proliferative activity and can be used to inoculate animals resulting in ascites in the case of abdominal growth, and in particular are used for fusion with immunised mouse splenocytes to form hybridomas.

CHO cells were cultivated for 3 days in culture medium. The resulting monolayer of cells was removed in trisodium citrate isotonic solution and sedimented by centrifugation for 10 minutes. The cells were resuspended in culture medium at a concentration of up to 66,000 cells per ml.

150 μl (cell count 10,000) of the CHO or Mα cell preparations which were obtained were used to inoculate microtiter wells and placed for 2 hours incubation in CO₂-containing atmosphere to allow adhesion to the well surface. Tolins were added to obtain a serial dilution in a volume of 100 μl. The cells were then cultivated for 5 days in an incubator with CO₂-atmosphere with daily observation.

The appearance of rounded cells and grain formation inside the cells is an indicator of cytotoxic action. These features were observed after only a single day of treatment. Subsequently no proliferation was observed. Phase microscopy allowed the cytotoxic action to be monitored. The cells have a rounded configuration and marked grain formation and loss of adhesion to the plastic is apparent. The threshold tolin concentration having cytotoxic activity can be determined on the basis of the last well in which cytotoxic effects are observed.

The results of a typical experiment are shown in FIG. 11.

2.9 Pathophysiological evaluation of changes in spleen and lymph node after infection with F. tularensis cells producing various tolins

Hamsters were infected with recombinant forms of F. tularensis to evaluate changes in the spleen and lymph node. Animals were injected under the skin with 10⁶-10⁸ cells and samples were collected 9 days post-infection. Infection with F. tularensis vaccine strain 15 NIIEG resulted in edema, necrotic nodules and centres of mucoid and fibroid swelling in hamster spleen. Infection with F. tularensis recombinant strain RB26 resulted in hamster spleen hyperplasia and showed nuclear polymorphism and hyperchromicity. Infection with F. tularensis recombinant strain RB26 resulted in hamster lymph node hyperplasia due to lymphoid and epithelioid cells. Beads were produced by lymphoid and epithelioid cells distal to the node necrosis band.

2.10 DNA-binding activity of unglycosylated tolins

Purified monomers were prepared as described in Example 1.2.3.

During the process of purification and concentration of the monomer, it was found that polymers were created. Polymers were identified as having a molecular weight of 116-158 kDa on non-denaturing SDS-PAGE. Under these non-denaturing conditions, monomers, dimers etc. were not identified although could be present at low concentrations.

DNA-binding tests were performed according to the method as described in Example 2.5 for determining nuclease activity, but using instead the unglycosylated polymer rather than tolins. The mixture was incubated at room temperature for 18 hours. Binding of the polymer to chromosomal DNA was assessed using scanning electron microscopy. As a control chromosomal DNA alone was examined as well as a mixture of tolin and chromosomal DNA. 2% uranyl acetate was used for contrast.

Results

Only the unglycosylated polymer was found to bind to chromosomal DNA whereas the tolin was found separate to the DNA.

### EXAMPLE 3

### Vaccine Activity

Three recombinant microorganisms (RM2, RM28 and RM32) were tested as vaccine candidates in white rats, white mice, golden hamsters and guinea pigs, which were challenged with the pathogenic bacteria Pseudomonas pseudomallei (C141).

The guinea pigs and white rats were infected with 1×10⁸ microorganisms per animal (RM28) and the golden hamsters and mice with 1×10⁶ microorganisms per animal (RM32).

Golden hamsters were challenged with 10× lethal dose (1× lethal dose equivalent to a single cell) of the virulent pathogenic bacteria. Guinea pigs, white rats and white mice were challenged with 100× lethal dose of the virulent pathogenic bacteria.

RM32 yielded 60% protection in guinea pigs (6 out of 10 animals survived compared to none of 9 animals in the control group). Average life span in the vaccinated group was 21 days compared to 18 days in the control group.

RM28 yielded 66% protection in golden hamsters (4 out of 6 animals survived compared to none of 5 animals in the control group). Average life span in the vaccinated group was 10 days compared to 6 days in the control group.

1. A bacterial protein monomer, preferaly glycosylated with at least the mossacharides glucose, xylose, rhatose axid ribose, which has a molecular weight of 12 to 25 kda, as asseded by denaturing SDS-page disk electrophoresis, and which in naturally ocurring from forms part of a bacterial glycoprotein polymer present in the capsule.of a bacterial cell containing the amino acid sequence:
```
Met Glu Leu Lys Leu Glu Asn Lys Gln Glu Ile ILe Asp Gln Leu
                                    5 10 15
Asn Lys Ile Leu Gln Leu Glu Met Ser Gly Ile Val Arg Tyr Thr
                                  20 25 30
His Tyr Ser Leu Met Ile Ile Gly His Asn Arg Ile Pro Ile Val
                                  35 40 45
Trp Ser Met Gln Ser Gln Ala Ser Glu Ser Leu Thr His Ala Thr
                                  50 55 60
Ala Ala Gly Glu Met Ile Thr His Phe Gly Gln His Pro Ser Leu
                                  65 70 75
Lye Ile Ala Asp Leu Aen Gln Thr Tyr Gln His Asn ILe Asn Asp
                                  80 85 90
Ile Leu Ile Glu Ser Leu Gln His Glu Lys Lys Ala Val Ser Ala
                                  95 100 105
Tyr Tyr Gln Leu Leu Lys Leu Val Asn Gly Lys Ser Ile Ile Leu
                                110 115 120
Glu Gln Tyr Ala Arg Lys Leu Ile Val Gln Gln Gln Thr His Ile
                                125 130 135
Gly Gln Val Gln Lys Met Leu Arg Lys Tyr
                                140 145
```
or a sequence which has more than 60%, preferably more than 89%, sequence homology thereto; or a functionally-equivalent variant with a sequence which has more than 60%, preferably more than 80% sequence homology thereto; or fragment or precursor thereof.

2. A bacterial protein monomer, preferably glycosylated with at least the monosaccharides glucose, xylose rharoge and ribose, which has a molecular weight of 12 to 25 kDa, as assesed by denaturing SDS-PAGE disk electrophoresis, and which in naturally ocurring form forms part of a bacterial glycoprotein polymer present in the capsule of a bacterial cell containing one or more of the amino acid sequences consisting of:
```
(i)
Xxx Asn Arg Gly Ala Val Arg Lys Val Leu Thr Thr Gly Leu Xxx
                                          5 10
Ala Xxx Ile
  15
(ii)
Xxx Asn Val Ser Glu Xxx Val Ser Ala Arg Ala Lys Glu Ala Asp
                                  5 10 15
Val Thr Xxx Glu Val Ala Ser Asn Thr Xxx Asp Ala Thr Ile Ala
                                  20 25 30
Ala Val Thr Xxx Ala Xxx Xxx Asn Xxx Xxx Ser Val Thr Leu Xxx
                                  35 40 45
Gly ; and
(iii)
Met Asn Lys Ser Glu Leu Val Ser Ala Ile Ala Lys Glu Ala Asp
                                    5 10 15
Val Thr Lys Glu Val Ala Ser Asn Thr Ile Asp Ala Thr Ile Ala
                                  20 25 30
Ala Val Thr Lys Ala Leu Lys Asn Gly Asp Ser
                                  35 40
```
or a sequence which exhibits more than 60%, preferably more than 80% sequence homology thereto, wherein “Xxx” denotes unknown or variable residues which in the latter case may be any amino acid; or a functionally-equivalent variant with a sequence which has more than 60%, preferably more than 80%, sequence homology thereto, or fragment or precursor thereof.

3. A bacterial protein polymer, preferably glycosylated, comprising at least 4 monomers, which may be the same or different, wherein at least one monomer, preferably all monomers are as defined in claim 1 or 2, and said polymer has a molecular weight of 116 to 158 kDa as assessed by non-denaturing SDS-PAGE by disk electrophoresi.

4. A bacterial protein polymer as claimed in claim 3 wherein said polymer is glycosylated and exhibits-nuclease activity on DNA and P samples in vitro.

5. A bacterial protein polymer as claimed in claim 3 or 4 wherein said polymer elutes at 150 mM NaCl from DEEM cellulose and elutes at 51-52% acetonitrile rom Nucleosil-C₁₈.

6. A nucleic acid molecule encoding a bacterial protein au defined in any one of claims 1 to 5, or a functionally-equivalent variant, derivative, fragment or precursor thereof.

7. A cloning or expression vector containing a nucleic acid molecule as defined in claim 6.

8. A transformed or transfected prokaryotic or eukaryotic host cell, or transgenic organism containing a nucleic acid molecule as defined in claim 6 or a cloning or expression vector as defined in claim 7.

9. A host cell as claimed in claim a wherein said prokaryotic cell is a microorganism corresponding to RTC16, RCC207, RM32, RM28, R5S, RN4, R1A, RM2, RB7, RB26, RC117, RVT-1 or RVT-2 deposited ar the Russian National Collection of Industrial Microorganisms (VKPM) under the Sudapest Treaty and given Accession numbers VKPM B-7673, VKPM B-7672, VKPM B-7671, VKPM B-7670 (deposited on Nov. 16, 1998), VKPM B-6853, VKPM B-6855, VKPM B-6852 (deposited on Aug. 8, 1994), VKPM B-7381, VKPM B-7383, VKPM B-7382, VKPM B-7384 (deposited on Apr. 8, 1997), VKPM-7776 and VKPM-7775 (deposited on May 7, 1999), respectively.

10. A method of isolating a bacterial protein, preferably glycoprotein, as claimed in ariy one of claims 1 to 5, wherein said method comprises cultyxing a host cell as defined in claim 8 or 9 under conditions whereby said bacterial protein is expressed and recovering said bacterial protein thus produced.

11. A method of isolating a bacterial protein, preferably glycoprotein, as defined in any one of claim 1 to 5, comprising at least the step of subjecting a crude extract of bacteria to enrichment and recovering the bacterial protein polymer-containing fractions by chromatography or gradient ultracentrifugation.

12. A method of isolating a bacterial protein, preferably glycoprotein, as defined in any one of claims 1 to 5, comprising at least the steps of preparing an extract of said bacteria, purifying said bacterial protein therefrom by binding said bacterial protein to an immobilized phase including a specific binding partner for the bacterial protein and subsequently eluting said bacterial protein from said immobilized phase.

13. A method as claimed in any one of claims 10 to 12 wherein said bacterial protein, preferably glycoprotein, is isolated from gram negative or gram positive bacteria, preferably bacteria of the genera Pteudoimonas (Atikholderia) or Mycobacterium.

14. A bacterial protein, preferably glycoprotein, obtainable by a method as defined in any one of claims 10 to 13.

15. A vaccine composition comprising one or more bacterial proteins, preferably glycoprotein polymers, as defined in any one of claims 1 to 5 or 14, or functionally-equivalent variants, derivatives, antigenic fragments or precursors thereof, together with at least one pharmaceutically acceptable carrier, diluent or excipient.

16. A vaccine composition as claimed in claim 16 comprising a host cell as defined in claim 8 or 9 wherein said bacterial protein is produced in vivo.

17. A method of stimulating an immune response against a bacterium in a human or non-human animal, comprising administering to said animal a vaccine composition as defined in claim 15 or 16 containing or expressing a bacterial protein, or functionally-equivalent variant, fragment or precursor thereof, from said bacterium or a related bacterium.

18. An antibody or antigen-binding fragment thereof which binds to a bacterial protein as defined in any one of claims 1 to 5 or 14.

19. A method of identifying the presence, or determining the amount, of a bacterium or part thereof in a sample, comprising at least the step of assessing the presence or amount of a bacterial protein as defined in any one of claims 1 to 5 or 14 or fragment thereof or nucleic acid molecule encoding said protein or fragment thereof in said sample.

20. A kit for identifying the presence, or determining the amount, of a particular bacterium or part thereof in a sample, comprising at least the following:
i) a signaling means comprising a label-carrying antibody binding to a bacterial protein as defined in any one of claims 1 to 5 or 14 a fragment thereof, specific to said bacterium, or a substrate appropriate to the enzymatic activity of said bacterial protein, or a labelled nucleic acid probe which binds to a nucleic acid molecule encoding a bacterial protein as defined in any one of claims 1 to 5 or 14 or fragment thereof.

21. A method of diagnosing infection of a human or non-human animal by a bacterium, wherein said method comprises at least the step of assessing the presence or amount of a bacterial protein as defined in any one of claims 1 to 5 or 14 or fragment thereof or nucleic acid molecules encoding said protein or fragment thereof in a sample from said human or non-human animal.

22. A method of diagnosing infection of a human or non-human animal by a bacterium by assessing the reaction of said animal to presentation of a bacterial protein as defined in any one of claims 1 to 5 or 14 obtainable from said bacterium.

23. A method of identifying a bacterial protein polymer of the invention suitable for use as an anti-proliferative, comprising at least the steps of a) growing said cells in the absence and presence of different bacterial protein polymers as defined in any one of claims 3 to 5 and b) comparing the number of live cells which remain after a time interval and c) identifying the bacterial protein polymer which inhibits cell proliferation to the greatest extent during said time interval.

24. The use of a bacterial protein polymez as defined in any one of claims 3 to 5 as an anti-proliferative agent or to alter the proliferation of cells.

25. A method of treating or preventing a condition associated with rapidly growing cells, preferably a tumour or leukemia, in a human or non-human animal comprising administering to said animal a bacterial protein polymer as defined in any one of claims 3 to 5 or a vaccine composition as defined in claim 15 or 16.

26. A method of diagnosing the presence or location of fast-growing cells, in a human or non-human animal, wherein said method comprises at least the step of assessing the association of a bacterial protein or fragment thereof as defined in any one of claims 1 to 5 with cells of said animal.

27. A method of cleaving nucleic acid molecules in vitro, wherein said nucleic acid material is contacted with a bacterial protein polymer as defined in any one of claims 3 to 5 for a time and a concentration appropriate to result in partial or complete cleavage of said nucleic acid molecules.


SEQUENCE LISTING (5 sequences)

SEQ ID NO: 1 - protein, 145 aa, Francisella tularensis
MELKLENKQEIIDQLNKILELEMSGIVRYTHYSLMIIGHNRIPIVWSMQSQASESLTHATAAGEMITHFGEHPSLKIADLNETYQHNINDILIESLEHEKKAVSAYYELLKLVNGKSIILEEYARKLIVEEETHIGEVEKMLRKY

SEQ ID NO: 2 - protein, 17 aa, Francisella tularensis
  Xaa represents any amino acid residue
XNGAVRKVLTTGLXAXI

SEQ ID NO: 3 - protein, 17 aa, Francisella tularensis
  Xaa represents any amino acis residue
XRGAVRKVLTTGLXAXI

SEQ ID NO: 4 - protein, 46 aa, Recombinant Bacteria
  Xaa represents any amino acid residue
XNVSEXVSARAKEADVTXEVASNTXDATIAAVTXAXXNXXSVTLXG

SEQ ID NO: 5 - protein, 41 aa, Francisella tularensis
MNKSELVSAIAKEADVTKEVASNTIDATIAAVTKALKNGDS